(19) United States
(12) Patent Application Publication
(10) Pub. No.: US 2002/0160393 A1
(43) Pub. Date: Oct. 31, 2002

(54) Double-stranded RNA-mediated gene suppression

(76) Inventors: Geoffrey P. Symonds, Rosebay (AU); Gregory C. Fanning, Bronte (AU); Gregory M. Arndt, Malabar (AU); Mitch Raponi, San Diego, CA (US); Nham Trieu Tran, Guildford (AU)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/035,098

(22) Filed: Dec. 28, 2001

Related U.S. Application Data
(60) Provisional application No. 60/258,733, filed on Dec. 28, 2000.
(60) Provisional application No. 60/258,731, filed on Dec. 28, 2000.

(30) Foreign Application Priority Data
Feb. 9, 2001 (AU) PR3028

(51) Int. Cl. C12Q 1/68; C07H 21/02; C12N 15/87

(52) U.S. Cl. 435/6; 536/23.1; 435/455

(57) ABSTRACT

The present invention relates to methods for modifying gene expression and in particular to methods for controlling gene expression in eukaryotic cells using double-stranded RNA (dsRNA), and to eukaryotic cell lines in which gene expression has been altered using the method. The invention also relates to compositions suitable for controlling gene expression and to methods of treatment which utilise such compositions.

This application claims priority from U.S Provisional Patent Application No. 60/258,733 filed Dec. 28, 2000 and entitled “Double-Stranded RNA and Related Molecules, Compositions and Methods; U.S. Provisional Application No. 60/258,731 filed Dec. 28, 2000 and entitled “Tat-based Methods for Facilitating Double-Stranded RNA-mediated Gene Suppression; and Australian Provisional Application No. PR3028 filed Feb. 9, 2001 and entitled Gene Suppression in Mammalian Cells.

### FIELD OF THE INVENTION

The present invention relates to methods for modifying gene expression and in particular to methods for controlling gene expression in eukaryotic cells using double-stranded RNA (dsRNA), and to eukaryotic cell lines in which gene expression has been altered using the method. The invention also relates to compositions suitable for controlling gene expression and to methods of treatment which utilise such compositions.

### BACKGROUND

The completion of the genomic sequence of a number of different organisms, including humans, has resulted in the identification of a large number of novel genes for which a biological function is not yet known. Therefore, there is a need to continue to develop effective methods for controlling the expression of specific genes, particularly in mammalian cell culture, and for studying the cellular role of these putative genes (Fields, 1997). Functional inactivation of a gene in organisms not readily amenable to gene disruption has been accomplished using gene constructs expressing sequences that encode antisense RNAs (van der Krol et al., 1988), homologous sense RNAs (Bunnell et al., 1990), ribozymes (Sarver et al., 1990) or dominant negative polypeptides (Herskowitz, 1988). The common feature of all of these forms of trans-acting genetic inhibitors is that they are derived from a target sequence. These techniques have their origins in the principle of “pathogen-derived resistance” which suggests that nucleotide sequences derived from a pathogen can be used to genetically modify a host to be resistant to that pathogen (Sanford, 1988). Identification of these gene-specific suppressors is time-consuming and usually requires extensive knowledge of either the domain structure of the protein or time-consuming screening of large numbers of candidate constructs encoding antisense RNAs, sense RNAs or ribozymes. In most instances knowledge of the structural and functional components of the target gene is required.

More recently, gene-specific double-stranded RNA has been used in some eukaryotic cell types for regulating the expression of specific genes (Fire et al., 1998, Nature 391, 860-811). The most common strategy is the generation of two complementary RNA strands in vitro, annealing of these strands to form dsRNA, and delivery of this synthesised dsRNA to the target cells. The original studies indicating that dsRNA could regulate specific gene expression demonstrated that this molecule was more effective than either antisense or sense RNA alone and that the mechanism of action of the dsRNA resulted in degradation of the target mRNA. To date, the application of dsRNA to regulate specific gene expression in mammalian cells has been restricted to the use of long and short synthetically derived dsRNAs.

The application of gene-expressed methods for generating dsRNA for gene suppression in human cells has not been successful. This is most likely due to the fact that differentiated somatic mammalian cells respond to exogenously delivered dsRNA with an interferon response (Marcus, 1983), which includes the activation of a dsRNA-responsive protein kinase (PKR) (Clemens, 1997). This enzyme phosphorylates and inactivates the translation initiation factor eIF2α, resulting in general translational arrest and eventually cell apoptosis. Thus, a priori evidence suggests that the differentiated mammalian cell may be recalcitrant to specific gene inactivation by exogenously-delivered dsRNA.

There is therefore a need for new methods to effectively and predictably alter the expression of a target gene in mammalian cells not only as a method to identify gene function but as a therapy for specific inhibition of protein expression.

The object of the present invention is to ameliorate at least some of the deficiencies of the prior art or to provide a useful alternative.

The foregoing and following description of, and references to, the prior art is provided so that the present invention may be more fully understood and appreciated in its technical context and in its significance. Unless clearly indicated to the contrary, however, this discussion is not, and should not be interpreted as, an express or implied admission that any of the prior art referred to is widely known or forms part of common general knowledge in the field.

### SUMMARY OF THE INVENTION

It has surprisingly and unexpectedly been found that RNA which has the potential to form intramolecular and/or intermolecular double-stranded RNA (“dsRNA”) , can be used effectively to modulate expression of a target gene in a cell, particularly in eukaryotic cells.

In a first embodiment, the invention relates to a double-stranded RNA complex comprising:

(a) a first ribonucleic acid molecule capable of hybridizing under physiological conditions to at least a portion of an mRNA molecule; and

(b) a second ribonucleic acid molecule wherein at least a portion of the second ribonucleic acid molecule is capable of hybridizing under physiological conditions to the first portion.

In one aspect of this embodiment, the first and second portions are separate ribonucleic acid molecules. In another aspect, the mRNA is encoded by a gene in a cell.

The invention also relates to a linear RNA molecule capable of forming a dsRNA complex wherein the RNA molecule comprises:

(a) a first portion that hybridizes to at least a portion of a mRNA molecule; and

(b) a second portion wherein at least part of the second portion is capable of hybridizing to the first portion to form a hairpin dsRNA complex.

Preferably the mRNA is encoded by a gene in a cell. This embodiment can further include a third portion of ribonucleic acid interposed between the first and second portions. This third portion can be useful in promoting hybridization between the first and second portion.

In either of these two embodiments an additional RNA portion of ribonucleic acid can be included that enhances the ability of dsRNA to alter transcription from the gene encoding the mRNA molecule. In one aspect this additional RNA portion encodes an RNA molecule and in another the additional RNA portion encodes a protein. As one example, the protein is Tat, other examples are detailed below.

Also, in either of these two embodiments the third portion of ribonucleic acid can further comprises at least one ribozyme and a target sequence recognizable by the ribozyme wherein the target sequence is not present in the first portion and the second portion. As a preferred strategy, although not required, the double-stranded RNA complex is formed upon hybridization of the first and second portion and the target sequence is cleaved by the hairpin dsRNA. Additionally, the third portion of ribonucleic acid further comprises an intron or a linker sequence.

In yet another embodiment of this invention, the invention relates to a linear RNA molecule capable of forming a dsRNA complex wherein the RNA molecule comprises:

(a) a first portion that comprises a region of RNA that is complementary to at least a portion of a mRNA molecule encoded by a gene;

(b) a second portion capable of hybridizing to at least part of the first portion; and

(c) a third portion positioned between the first and second portions to permit the first and second portions to hybridize with one another.

In a preferred aspect of this third embodiment, the third portion comprises at least one ribozyme and a target sequence recognized by the ribozyme wherein the target sequence is not present in the first or second portion. In another aspect the second sequence can further comprise a polyadenylation signal. The third sequence can include one ribozyme or a plurality of ribozymes and target sequences capable of cleavage thereby.

In another embodiment of this invention, the invention relates to a linear RNA molecule capable of forming a dsRNA complex wherein the RNA molecule comprises:

(a) a first portion that hybridizes to at least a portion of a mRNA molecule encoded by a gene; and

(b) a second portion wherein at least part of the second portion is capable of hybridizing to the first portion and wherein the second portion comprises a polyadenylation signal and a ribozyme positioned between the part of the second portion capable of hybridizing to the first portion and the polyadenylation signal

wherein the ribozyme is capable of removing the polyadenylation signal.

In a preferred aspect of this embodiment, the ribozyme is a cis-acting hammerhead ribozyme.

These embodiments may also take the form of DNA, such that the DNA is capable of generating the RNA molecules of this invention using the transcriptional machinery, for example, available in a cell or in cell lysates preparations. The RNA molecules may be provided to a cell as a single DNA molecule or as two or more DNA molecules.

In a further embodiment there is provided a double-stranded RNA complex, which RNA comprises,

(A) a first sequence which, under hybridizing conditions, hybridizes to at least a portion of an mRNA molecule encoded by a gene; and

(B) a second sequence which, under hybridizing conditions, hybridizes to the first sequence; and the first and second sequences are part of independent linear RNA molecules.

In another embodiment there is provided a linear RNA molecule for forming a double-stranded RNA complex, which RNA comprises,

(A) a first sequence which, under hybridizing conditions, hybridizes to at least a portion of an mRNA molecule encoded by a gene; and

(B) a second sequence which, under hybridizing conditions, hybridizes to the first sequence; and the complex between sequences one and two produces an artificial hairpin dsRNA. In another aspect there is provided a linear RNA molecule for forming a double-stranded RNA complex, which RNA molecule comprises,

(a) a portion encoding an RNA or protein that enhances the specific activity of dsRNA; and

(b) a portion for forming a double-stranded RNA complex, which portion comprises

(i) a first sequence which, under hybridizing conditions, hybridizes to at least a portion of an mRNA molecule encoded by the gene;

(ii) a second sequence which, under hybridizing conditions, hybridizes to the first sequence; and

(iii) a third sequence situated between the first and second sequences so as to permit the first and second sequences to hybridize with each other,

whereby, under hybridizing conditions, the portion (b) forms a double-stranded RNA complex upon hybridization between the first and second sequences.

In a preferred embodiment the protein that enhances the specific activity of dsRNA would be the HIV Tat protein.

In a preferred embodiment the third sequence comprises (i) a ribozyme and (ii) a target sequence specifically recognized by the ribozyme and absent in the first and second sequences, whereby the complex-forming portion forms a double-stranded RNA complex upon hybridization between the first and second sequences and the target sequence is cleaved by the ribozyme. The third sequence may also comprises a plurality of ribozymes and target sequences cleaved thereby.

In further preferred embodiments the third sequence comprises an intron, a portion of the target sequence not contained in either of sequences 1 or 2, or a linker sequence.

In another aspect the invention provides a linear RNA molecule for forming a double-stranded RNA complex, which RNA molecule comprises

(a) a first sequence which, under hybridizing conditions, hybridizes to at least a portion of an mRNA molecule encoded by a gene;

(b) a second sequence which, under hybridizing conditions, hybridizes to the first sequence; and

(c) a third sequence situated between the first and second sequences so as to permit the first and second sequences to hybridize with each other, which third sequence comprises (i) a ribozyme and (ii) a target sequence which is specifically recognized by the ribozyme and is absent in the first and second sequences,

whereby, under hybridizing conditions, the RNA molecule forms a double-stranded RNA complex upon hybridization between the first and second sequences and the target sequence is cleaved by the ribozyme.

The third sequence in this embodiment of the invention may also comprise a plurality of ribozymes and target sequences cleaved thereby.

In another aspect there is provided a double-stranded RNA complex, which RNA comprises,

(B) a first sequence which, under hybridizing conditions, hybridizes to at least a portion of an mRNA molecule encoded by a gene; and

(C) a second sequence which, under hybridizing conditions, hybridizes to the first sequence; and the second sequence contains at its 3′ end, between the end of the region of complementarity with the first sequence and the polyadenylation signal, a cis-acting hammerhead ribozyme that can cleave within this same region and remove the polyadenylation signal.

This embodiment of the invention utilises a ribozyme to cleave the polyadenylation signal of the RNA molecule, thus retaining the RNA molecule and/or dsRNA in the nucleus.

The ribozyme may be any ribozyme as described in the literature referred to herein but preferred is a hammerhead ribozyme.

The RNA of the present invention may be a single molecule or may be more than one RNA molecule. When the RNA is a single molecule, the dsRNA may be formed by intramolecular RNA bonding. In embodiments where more than one RNA molecule is used, the dsRNA may be formed by intermolecular RNA bonding.

The invention also provides DNA molecules which encode the RNA molecules capable of forming dsRNA. Such a DNA molecule may be a single DNA molecule which, when introduced into a cell, gives rise to a single RNA molecule capable of forming intramolecular dsRNA. However it will be understood from the following description that more than one DNA molecule may be introduced into a cell, either simultaneously or sequentially, to give rise to two or more RNA molecules capable of forming intermolecular dsRNA. Typically the two RNA sequences capable of forming dsRNA, whether intra or intermolecularly, are at least in part sense and at least in part antisense sequences of a gene or nucleic acid sequence whose expression is to be suppressed. In preferred embodiments constructs comprising DNA which encodes the RNA capable of forming dsRNA are used to produce RNA in a cell.

The invention provides vectors comprising RNA or DNA molecules of the present invention, as well as cells comprising RNA or DNA molecules, or vectors comprising such molecules. Preferably, cells are mammalian cells and even more preferably they are human cells. It will be clear to the skilled addressee that the cells may be somatic, undifferentiated, dedifferentiated neoplastic, chimera cells or transgenic animal cells. The cells may, of course, be neoplastic cells.

It will also be clear to the person skilled in the art that the cells may be in vitro cultured cells or may be in situ and that the method has in vivo and ex vivo therapeutic applications.

Preferably, the RNA is encoded by a gene and said gene is transcribed in said cell and more preferably, the gene is delivered to said cell by means of a vector. Most preferably, the vector is a plasmid, adenovirus, adeno-associated virus, or retrovirus. In a preferred embodiment, the plasmid is an episomal plasmid. However, the invention is not limited to these types of vectors and the skilled addressee will be able to identify other suitable vectors.

It will be clear to the skilled addressee in light of the preceding discussion that any mechanism of introducing RNA which has the potential to form double-stranded RNA into a cell, and particularly into the cytoplasm or nucleus of the cell, will be useful in the present invention. As such, it is contemplated, that in certain cases, it may be useful to introduce RNA by, for example, vectors encoding the RNA, microinjection or by vesicle delivery and it will also be clear that the RNA may be in either be in single or double-stranded form at the time of introducing it into the cell. The RNA may, therefore be synthesised outside said cell by standard techniques.

Preferably, the RNA is retained within the nucleus of said cell. In one embodiment, the RNA is retained within the nucleus of said cell by deletion or cleavage of the polyadenylation signal. Cleavage of the polyadenylation signal from the RNA may be achieved by a cis-acting ribozyme or by any other suitable means.

In another aspect the invention provides a method of suppressing expression of a specified gene or a specified nucleic acid sequence in a eukaryotic cell comprising introducing into said cell an RNA molecule of the present invention, or a DNA molecule of the present invention, wherein said RNA molecule comprises first and second sequences corresponding to sense and antisense sequences with respect to the specified gene or the specified nucleic acid sequence and wherein said DNA molecule comprises sequences which encode first and second RNA molecules corresponding to sense and antisense sequences with respect to the specified gene or the specified nucleic acid sequence.

In another aspect the invention provides a mammalian cell in which a specified gene or a specified nucleic acid sequence has been suppressed by a method of the present invention.

In another aspect the invention provides a method of modulating expression of a gene or a nucleic acid sequence in mammalian cells including exposing said cells to medium in which mammalian cell of the present invention has been grown.

In yet another aspect the present invention provides a method of determining the function of a gene or a nucleic acid sequence including suppressing expression of the gene or nucleic acid sequence by a method of the present invention.

In a further aspect the present invention provides a method of determining the function of a protein by suppressing expression of the gene encoding the protein by a method of the present invention.

In another aspect the present invention provides a method of modulating a cellular response wherein said response is due either directly or indirectly to the expression of a gene or nucleic acid sequence and wherein expression of said gene or nucleic acid sequence is suppressed by a method of the present invention.

In yet another aspect the invention provides a composition for use in inhibiting the expression of a gene in a eukaryotic cell comprising

(a) an RNA molecule encoding HIV Tat protein; and

(b) a linear RNA molecule for forming a double-stranded RNA complex, which RNA molecule comprises

(i) a first sequence which, under hybridizing conditions, hybridizes to at least a portion of an mRNA molecule encoded by the gene;

(ii) a second sequence which, under hybridizing conditions, hybridizes to the first sequence; and

(iii) a third sequence situated between the first and second sequences so as to permit the first and second sequences to hybridize with each other,

whereby, under hybridizing conditions, the RNA molecule forms a double-stranded RNA complex upon hybridization between the first and second sequences.

In a further aspect the invention provides a composition for use in inhibiting the expression of a gene in a eukaryotic cell comprising

(a) a DNA molecule encoding HIV Tat protein; and

(b) a DNA molecule encoding a linear RNA molecule for forming a double-stranded RNA complex, which RNA molecule comprises

(i) a first sequence which, under hybridizing conditions, hybridizes to at least a portion of an mRNA molecule encoded by the gene;

(ii) a second sequence which, under hybridizing conditions, hybridizes to the first sequence; and

(iii) a third sequence situated between the first and second sequences so as to permit the first and second sequences to hybridize with each other, whereby, under hybridizing conditions, the RNA molecule forms a double-stranded RNA complex upon hybridization between the first and second sequences.

In a further aspect the present invention provides a method of treating a disorder resulting either directly or indirectly from expression of a gene or nucleic acid sequence wherein expression of said gene or nucleic acid sequence is suppressed by a method of the present invention.

### BRIEF DESCRIPTION OF THE FIGURES

FIG. 1. Reduction in destabilised green fluorescent protein (dEGFP)-mediated cell fluorescence in human embryonic kidney cells co-expressing sense and antisense dEGFP RNAs. FIG. 1A provides a schematic representation of the dEGFP target gene, sense genes and antisense genes used in Example 2. The integrated structure of the dEGFP target gene in the dEGFP-expressing cell line is indicated at the top of the figure. The dEGFP open reading frame (ORF) is under control of the CMV immediate early promoter and the SV40 polyadenylation signal. The sense and antisense dEGFP genes contained on episomal plasmids are indicated with the designation of each expression plasmid indicated at the left. The downward arrow indicates a single base change converting the ATG start codon in the dEGFP ORF to a CTG. The direction of the horizontal arrows indicates the natural direction of transcription. Other abbreviations are as follows: EF-1α, elongation factor 1α promoter; Pur^{R}, puromycin-N-acetyl transferase; RSV, Rous sarcoma virus long terminal repeat; Hyg^{R}, hygromycin B phosphotransferase; dEGFP ORF, dEGFP open reading frame. Each of the sense and antisense genes is shown linked with the selectable marker resident on the episome. FIG. 1B illustrates the effect of co-expressing sense and antisense dEGFP RNAs on dEGFP-mediated cell fluorescence. Two to three independent pooled populations for the indicated co-transfected plasmids were assayed for dEGFP-mediated cell fluorescence after growth to three different stages of confluence. The histograms represent the average geometric mean fluorescence and the error bars indicate the standard deviation. The legend describing each of the co-transfected populations is as follows: white-filled box: pREP7+pEAK10(JJR); black-filled box: pR7ctgES+pJEas; dot-filled box: pREP7+pJEas; hatched box: pJctgES+pR7ctgEas; diamond-filled box: pjctgES+pREP7; and brick-filled box: pEAK10(JJR)+pR7ctgEas. The abbreviations used are as in FIG. 1A.

FIG. 2. dEGFP mRNA steady-state levels in human embryonic kidney cells co-expressing sense and antisense dEGFP RNAs. FIG. 2 illustrates a quantitative analysis of the level of dEGFP mRNA relative to the 18S rRNA. The steady-state level of dEGFP target mRNA is expressed relative to the level of 18S rRNA for each of the co-transfected populations as indicated.

FIG. 3. dEGFP protein levels in human embryonic kidney cells co-expressing sense and antisense dEGFP RNAs. FIG. 3 provides a quantitative analysis of the level of dEGFP protein relative to β-actin. Each histogram represents the ratio of the dEGFP protein to the β-actin protein as determined by Western blot analysis.

FIG. 4. Suppression of dEGFP-mediated cell fluorescence by dsRNA conditioned medium. FIG. 4A provides an overview of a culture medium transfer experiment according to this invention. FIG. 4B illustrates results from an experiment according to FIG. 4A wherein suppression of dEGFP-mediated cell fluorescence by dsRNA conditioned medium derived from cells co-expressing sense and antisense dEGFP RNA is demonstrated. The code for the different histograms is shown at the bottom of the diagram. FIG. 4C provides a quantitative analysis of dEGFP and p53 protein levels relative to β-actin protein levels in control cells exposed to medium derived from control cells, DMEM medium, or medium from cells co-expressing sense and antisense dEGFP RNAs.

FIG. 5. Suppression of dEGFP-mediated cell fluorescence by expression of dsRNA from an inverted repeat plasmid. FIG. 5A is a schematic representation of the expression cassettes contained on the inverted repeat plasmids used in Example 4. Each of the three cassettes used to generate dEGFP-specific dsRNA is indicated. All of these inverted repeat genes are under control of the conditional ecdysone-inducible promoter (represented by HSP). The synthetic intervening sequence (IVS) is shown in the first two cassettes. The arrows indicate the normal direction of transcription. Each of these expression cassettes resides on an episomal plasmid containing the RFP gene. FIG. 5B illustrates the effect of expressing dEGFP-specific inverted repeat dsRNAs on dEGFP-mediated cell fluorescence. The level of dEGFP-mediated cell fluorescence relative to the control vector in the plasmid transfected population (RFP+) at 48 hours post-electroporation and 24 hours following the addition of ponasterone A (10 μM). The ‘vehicle’, as indicated by the white-filled histograms, is ethanol alone, while the black-filled histograms denote addition of the inducer ponasterone A.

FIG. 6. Effect of a cis-acting ribozyme on nuclear localisation of sense RNA. FIG. 6A is a schematic illustrating expression cassettes used in Example 6. Each of the reporter cassettes was used to test the efficacy of a cis-acting hammerhead ribozyme for localising sense GFP RNA inside the nucleus. The abbreviations are as indicated in FIG. 1A, with the exception of GFP, which represents green fluorescent protein, and RBZ which represents the sequence encoding the hammerhead ribozyme. FIG. 6B illustrates results from experiments to determine the effect of a cis-acting ribozyme on the nuclear localisation of sense RNA using the constructs of FIG. 6A and measuring the level of GFP-mediated cell fluorescence. All values are expressed as a percentage of the control cells.

FIG. 7. A proposed mechanism for dsRNA-mediated gene suppression. This figure illustrates a proposed mechanism for dsRNA-mediated gene suppression, in which proteins bind to dsRNA and initiate cleavage, resulting in 21-23-mers. The protein-bound fragments then go through an amplification step (presumably by the implicated RNA polymerases) and hybridize to the target mRNA. Either the physical anti-sense block prevents transcription or, more likely, further proteins are sequestered and cleavage of the target RNA occurs.

FIG. 8. Alternative mechanisms for the formation of dsRNA. This figure illustrates the formation of dsRNA according to one of the preferred embodiments of this invention. The first mechanism (FIG. 8A) involves the cloning of an intervening sequence that, upon transcription, forms a loop as the complementary sequences bind. The second mechanism (FIG. 8B) involves the inclusion of an intron with a splice donor/splice acceptor site such that, upon transcription, the cell machinery will splice out the intron leaving a hairpin RNA molecule homologous to the target sequence. The third mechanism (FIG. 8C), which is the subject of the instant invention, involves the inclusion of an intervening sequence that is flanked by ribozymes such that, upon transcription, the ribozymes excise the intervening sequence, leaving a dsRNA that is homologous to the target mRNA.

FIG. 9 illustrates exemplary retroviral constructs encoding HIV-specific dsRNA. Example A is an illustration of a retroviral vector composed of a retroviral LTR, a drug resistance gene such as Neomycin Phosphotransferase, a first sequence such as H5, optionally a third sequence, such as an intervening sequence, and a second sequence such as ASH5, and a second LTR; Example B is an illustration of a retroviral vector composed of a retroviral LTR that also contains an inducible element responsive to a host, chemical or viral factor, such as the HIV TAR sequence which binds to Tat to enhance transcription. The vector further includes a drug resistance gene such as Neomycin Phosphotransferase, a first sequence such as H5, a third sequence and a second sequence such as ASH5, and a second LTR. In this construct the check-filled box in the 5′ LTR represents an element that is responsive to Tat; Example C illustrates a retroviral vector composed of a retroviral LTR, a sequence encoding a protein that enhances the activity of the dsRNA such as the Tat protein, a first sequence such as H5, a third sequence, a second sequence as ASH5, an internal promoter such as the SV40 early/Late promoter, a drug resistance gene such as Neomycin Phosphotransferase, and a second LTR. The hatched box in all three constructs represents the third sequence referred to in Example 6.

### DESCRIPTION OF THE PREFERRED EMBODIMENTS

Definitions

The term “catalytic region” of a nucleic acid molecule, “catalytic nucleic acid molecule”, “catalytic nucleic acid”, and “catalytic nucleic acid sequence” as used herein are equivalent, and each shall mean a nucleic acid molecule which specifically recognizes a distinct substrate and catalyzes the chemical modification of this substrate.

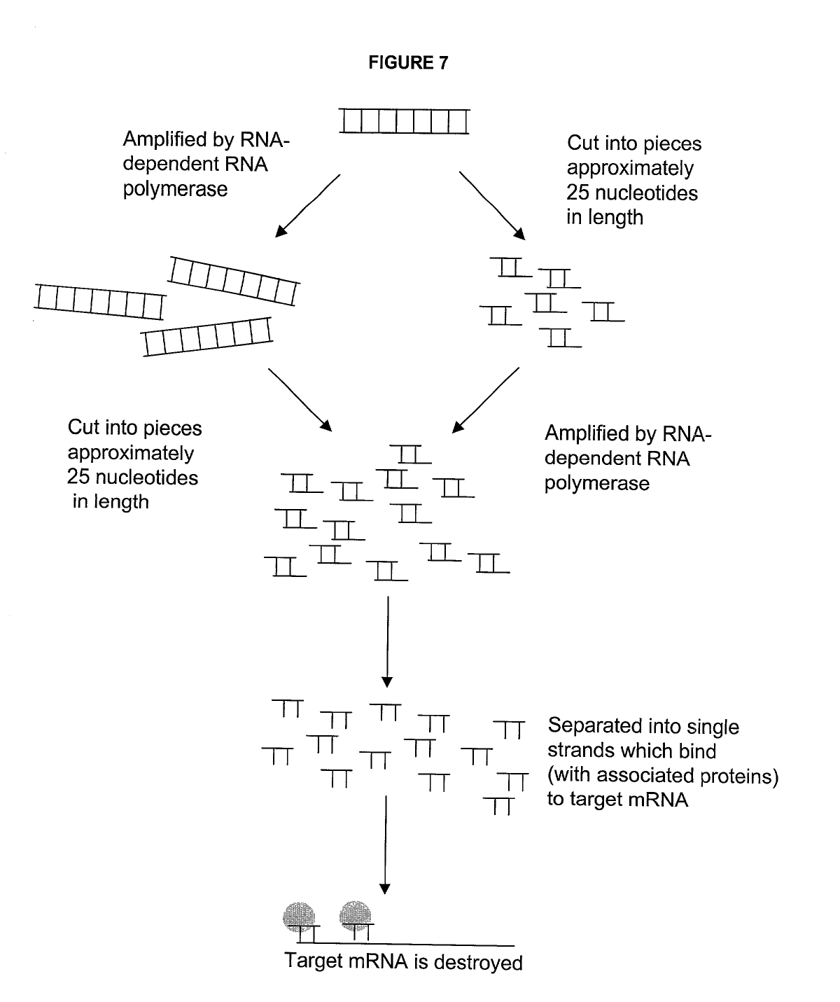

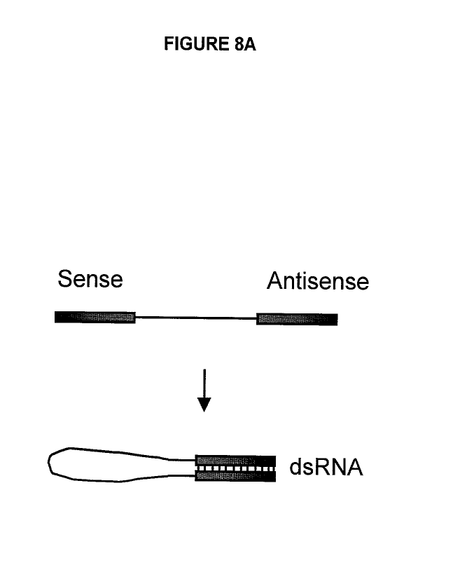

The term “double-stranded RNA complex”, “double-stranded RNA”, “dsRNA complex” and “dsRNA” as used herein are equivalent, and each shall mean a complex formed either (a) by two linear molecules of RNA, wherein at least a portion of the sequence of one molecule is complementary to, and is capable of or has hybridized to, at least a portion of the sequence of the other RNA molecule, or (b) by two portions of a linear RNA molecule which are complementary to, and are capable of or have therefore hybridized to, each other. Some of the mechanisms for the formation of dsRNA are shown in FIG. 7.

The term “hybridizing conditions” as used herein shall mean conditions permitting hybridization between two complementary strands of RNA having a length of at least seven nucleotides. Hybridizing conditions are well known in the art, and include, without limitation, physiological conditions, such as, but not limited to, intracellular physiological conditions.

The term “inhibiting” or “limiting” a disease, condition or disorder shall refer to a reduction in the likelihood of the onset or a disease, condition or disorder or the prevention of onset or the delay of onset of a disorder entirely. Alternatively, the terms shall also refer to a reduction in the intensity or severity of a particular disease, condition or disorder.

The term “suppressing” the expression of a gene in a eukaryotic cell refers to a process for lessening or reducing the degree to which a particular gene is expressed, or preferably, preventing or inhibiting such expression entirely.

The term “introducing” a dsRNA complex into a cell shall mean causing such complex to become present in the cell. This presence may come about through delivery into the cell of a dsRNA complex already formed outside the cell or, alternatively, through delivery into the cell of one of the instant nucleic acid molecules, either RNA or DNA, which, once in the cell, gives rise to a dsRNA complex.

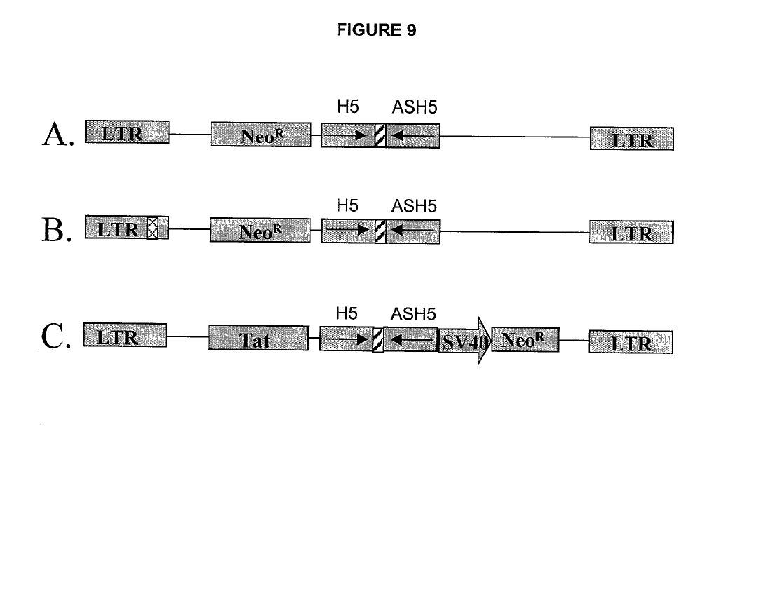

The term “intervening sequence” or “IVS” as used herein refers generally to the diagonally-hatched sequence as provided in FIG. 9. The sequence is preferably unrelated to the first and second sequence and can be an intron containing a splice donor and splice acceptor sequence or an intron containing multiple ribozymes.

The term “nucleic acid molecule” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

The term “ribozyme” as used herein shall refer to a catalytic nucleic acid molecule which is RNA or whose catalytic component is RNA, and which specifically recognizes and cleaves a distinct target nucleic acid sequence (also referred to herein as a “target” or “target sequence”) , which can be either DNA or RNA. Each ribozyme has a catalytic component (also referred to as a “catalytic domain”) and a target sequence-binding component consisting of two binding domains, one on either side of the catalytic domain. Ribozymes are described generally in [Sun et al (2000)]. In a preferred embodiment, the ribozyme is a hammerhead ribozyme.

The term “subject” as used herein shall refer to an animal, including, but not limited to a primate, mouse, rat, guinea pig or rabbit. In a preferred embodiment, the subject is a human.

The term “substrate” as used herein refers to a molecule that is specifically recognized and modified by a catalytic nucleic acid molecule.

The term “treating” a disorder as used herein shall mean slowing, inhibiting, stopping or reversing of the progression of a disorder. In a preferred embodiment, “treating” a disorder refers to reversing the progression of a disorder, ideally to the point of eliminating the disorder itself. As used herein, “ameliorating” a disorder and “treating” a disorder are used interchangeably. The term is also used in conjunction with terms “prophylactic” and “therapeutic” to more clearly differentiate between preventive and curative treatment.

The term “HIV Tat protein”, “Tat protein” and “Tat” are interchangeably used herein and refers to any of (a) the HIV protein comprising the amino acid sequence met-glu-pro-val-asp-pro-arg-leu-glu-pro-trp-lys-his-pro-gly-ser-gln-pro-lys-thr-ala-cys-thr

-asn-cys-tyr-cys-lys-lys-cys-cys-phe-his-cys-gln-val-cys-phe-ile-thr-lys-ala-leu-gly-ile-ser-tyr-gly-arg-lys-lys-arg-arg-gln-arg-arg-arg-pro-pro-gln-gly-ser-gln-thr-his-gln-val-ser-leu-ser-lys-gln-pro-thr-ser-gln-ser-arg-gly-asp-pro-thr-gly-pro-lys-glu, (b) HIV protein having the amino acid sequence tyr-gly-arg-lys-lys-arg-arg-gln-arg-arg-arg, and (c) all naturally occurring variants of proteins (a) and (b). Naturally occurring variants of HIV protein sequences can be found, inter alia, in Genbank and the Los Alamos HIV Database, both databases and naturally occurring variants being well known in the art.

The present invention relates to methods of controlling the expression of known genes or known nucleic acid sequences in eukaryotic cells using sense and antisense RNA sequences (with respect to the gene or nucleic acid sequence) capable of forming double-stranded RNA complexes. That is, the RNA molecules of this invention are capable of forming double stranded RNA and are capable of binding to a portion of a genome, to exogenous DNA or to an RNA molecule, preferably mRNA within a cell. Preferably the sense and antisense RNA sequences are encoded by one or more DNA molecules the expression of which gives rise to the RNA sequences capable of forming intramolecular or intermolecular double-stranded RNA (“dsRNA”) , thereby suppressing the expression of the gene or nucleic acid sequence. However, one or more RNA molecules may be introduced into a cell, wherein intramolecular (dsRNA formed using a single RNA strand) or intermolecular dsRNA (ds RNA formed using two or more separate RNA strands) is formed within the cells, or the dsRNA may be introduced into a cell as a preformed complex.

Thus, the invention also relates to RNA molecules for forming dsRNA, to DNA molecules encoding the RNA molecules for forming dsRNA, to vectors and cells comprising such molecules, to compositions comprising the molecules and vectors, and to prophylactic and therapeutic methods for administering the RNA molecules, the DNA molecules and the dsRNA. In certain embodiments the invention employs ribozyme-containing RNA molecules to generate dsRNA complexes, thereby overcoming certain known difficulties associated with generating dsRNA. In other embodiments the invention is based on the ability of a portion of the RNA molecule to encode an RNA or protein that enhances specific activity of ds RNA. One example of this specific activity enhancing portion of the RNA molecule is a portion of the molecule encoding the HIV Tat protein to inhibit the cellular breakdown of dsRNA complexes. Such a portion is additionally useful in treating disorders such as HIV infection. In yet other embodiments the invention employs ribozyme-containing RNA molecules to remove polyadenylation signals, thus preventing or minimising release of the RNA molecule from the nucleus of a cell. Other embodiments of the invention make use of co-transfection procedures for introduction of multiple RNA or DNA molecules to facilitate intermolecular dsRNA formation, and the use of detectable markers to facilitate identification of suppressed genes or nucleic acid sequences.

The instant molecules (RNA and DNA), compositions and methods have numerous uses for treating or inhibiting the onset of disorders which would be ameliorated by suppressing the expression of known genes.

In one aspect there is provided a double-stranded RNA complex, which RNA comprises, a first ribonucleic acid molecule capable of hybridizing under physiological conditions to at least a portion of an mRNA molecule, and a second ribonucleic acid molecule wherein at least a portion of the second ribonucleic acid molecule is capable of hybridizing under physiological conditions to the first portion. Preferably the first and second portions are on separate ribonucleic acid molecules. The molecules are capable of hybridization at physiological conditions, such as those existing within a cell and upon hybridization the first and second portions form a double stranded RNA molecule.

This type of RNA molecule could be obtained within a cell through the introduction of a single expression plasmid having two separate expression cassettes encoding the complementary RNAs, or more preferably by introducing two expression vectors each encoding one of the two linear RNA molecules. The generation of the two linear RNA molecules can most easily be achieved by constructing a two DNA molecules each containing (a) a promoter, operative in the cell, (b) a DNA region capable of being transcribed into an RNA molecule with a nucleotide sequence of at least 20 nucleotides identical with at least part of the nucleotide sequence of the nucleic acid of interest, or an antisense sequence wherein the RNA molecule is capable of forming a double-stranded RNA by base pairing between the regions with sense and antisense nucleotide sequence resulting in an intermolecular dsRNA structure, and (c) a DNA region encoding transcription termination and polyadenylation signals. Preferred embodiments for the different structural and functional characteristics, such as length and sequence of the antisense and sense regions, of this method are described elsewhere in the specification.

In another aspect there is provided a linear RNA molecule for forming a double-stranded RNA complex, which RNA comprises a first portion capable of hybridizing to at least a portion of a mRNA molecule, preferably within a cell and a second portion wherein at least part of the second portion is capable of hybridizing to the first portion to form a hairpin dsRNA complex.

In one embodiment the RNA portions are on a single linear RNA molecule and through intramolecular hybridization a dsRNA complex is formed. The distance between the first and second portions can vary from no sequence between the first and second portions or where a restriction enzyme recognition site (less than or equal to eight base pairs) is positioned between the portions or larger regions. The term hairpin dsRNA refers to dsRNA molecules that are capable of folding back on themselves such that a hairpin like structure of nonhomology is formed between the regions of homology. This dsRNA complex would preferably be formed through expression from an expression vector. This could most easily be achieved by constructing a chimeric DNA molecule containing (a) a promoter, operative in the cell, (b) a DNA region capable of being transcribed into an RNA molecule with a nucleotide sequence of at least 20 nucleotides identical with at least part of the nucleotide sequence of the nucleic acid of interest, and an antisense sequence wherein the RNA molecule is capable of forming a dsRNA by base pairing between the regions with sense and antisense nucleotide sequence resulting in a hairpin dsRNA structure, and (c) a DNA region encoding transcription termination and polyadenylation signals. In the preferred embodiment the RNA molecule transcribed from the chimeric gene, consists essentially of the hairpin RNA, and the order of the sense and antisense sequences is not essential.

In another embodiment there is provided a linear RNA molecule for forming a double-stranded RNA complex, which RNA molecule further comprises a portion encoding an RNA or protein that enhances the specific activity of dsRNA (i.e., it enhances the ability of the dsRNA to alter transcription from the gene encoding the mRNA molecule). In this variation a double stranded RNA molecule is formed and an enhancing element, preferably a protein is encoded on the RNA or as a separate RNA sequence to promote binding of the dsRNA to its specific target.

In a preferred embodiment a linear RNA molecule containing a portion encoding a protein capable is provided to enhance the efficiency of specific gene regulation using dsRNA. The dsRNA also includes a portion capable of binding the dsRNA specifically to its target sequence. The protein component could be any variety of proteins including, but not limited to viral proteins capable of modulating the global mammalian cell response to dsRNA, and would include but not be restricted to, mammalian viral proteins (vaccinia virus early protein E3L, reovirus p3 protein, vaccinia virus pK3, HIV-1 Tat) or cellular proteins (PKR dominant negative proteins, p58, and oncogenes such as v-erbB, sos or activated ras). In addition the protein component could be any enzyme component of the host protein complex that acts specifically on dsRNA to enhance the efficacy of the dsRNA in controlling specific gene expression. In a preferred embodiment the protein that enhances the specific activity of dsRNA would be the HIV Tat protein. The RNA components capable of enhancing specific regulation by dsRNA would include, but not be restricted to, short viral or cellular dsRNAs (such as adenovirus VAI, HIV-1 TAR, EBER-1, and Alu RNAs).

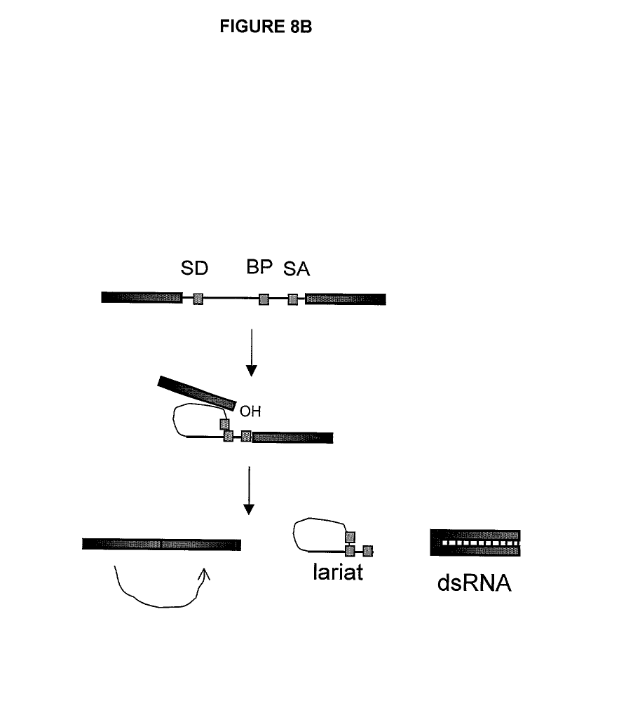

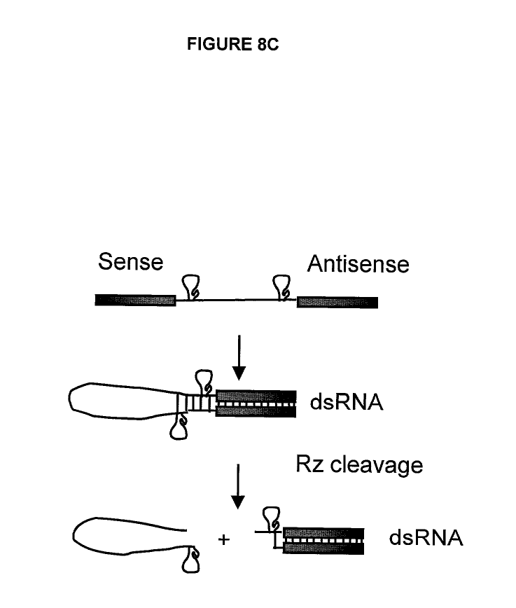

In yet another preferred embodiment a third portion on a linear RNA molecule is provided that includes (i) a ribozyme and (ii) a target sequence specifically recognized by the ribozyme and absent in the first and second sequences, whereby the complex-forming portion forms a double-stranded RNA complex upon hybridization between the first and second sequences and the target sequence is cleaved by the ribozyme. The third sequence may also comprise a plurality of ribozymes and target sequences cleaved thereby. In a preferred embodiment illustrated in FIG. 8, an intervening sequence is flanked by ribozymes such that, upon transcription, the ribozymes excise the intervening sequence, leaving a dsRNA that is homologous to the target mRNA. In further preferred embodiments the third sequence comprises an intron, a portion of the target sequence not contained in either of sequences 1 or 2, or a linker sequence.

The linear dsRNA complex would preferably be formed through expression from an expression vector, and preferably an episomal plasmid or retroviral vector. This could most easily be achieved by constructing a chimeric DNA molecule containing (a) a promoter, operative in the cell, (b) a DNA region encoding a protein capable of enhancing dsRNA specific activity (c) a DNA region capable of being transcribed into an RNA molecule with a nucleotide sequence of at least 20 nucleotides identical with at least part of the nucleotide sequence of the nucleic acid of interest, and an antisense sequence wherein the RNA molecule is capable of forming a dsRNA by base pairing between the regions with sense and antisense nucleotide sequence resulting in a intramolecular dsRNA structure, (d) a third DNA sequence between the sense and antisense sequences in (c), (e) a DNA region encoding a positive selectable marker and (e) a DNA region encoding transcription termination and polyadenylation signals. In the preferred embodiment the RNA molecule transcribed from the chimeric gene comprises a region encoding a protein capable of enhancing the specific action of dsRNA and a portion capable of forming intramolecular dsRNA specific to a target sequence. Those skilled in the art will realise that portion (a) above can be expressed within the same linear RNA as portion (b), or can be co-expressed with portion (b) from a separate chimeric DNA molecule containing (a) a promoter, operative in the cell, (b) a DNA region encoding a protein capable of enhancing dsRNA specific activity, and (c) a DNA region encoding transcription termination and polyadenylation signals. In one embodiment this invention provides a linear RNA molecule for forming a double-stranded RNA complex, which RNA molecule comprises,

(a) a portion encoding HIV Tat protein; and

(b) a portion for forming a double-stranded RNA complex, which portion comprises

(i) a first sequence which, under hybridizing conditions, hybridizes to at least a portion of an mRNA molecule encoded by a gene or a nucleic acid;

(ii) a second sequence which, under hybridizing conditions, hybridizes to the first sequence; and

(iii) a third sequence situated between the first and second sequences so as to permit the first and second sequences to hybridize with each other, whereby, under hybridizing conditions, the portion (b) forms a double-stranded RNA complex upon hybridization between the first and second sequences.

The invention further provides a linear RNA molecule for forming a double-stranded RNA complex, which RNA molecule comprises

(a) a first sequence which, under hybridizing conditions, hybridizes to at least a portion of an mRNA molecule encoded by a gene or a nucleic acid;

(b) a second sequence which, under hybridizing conditions, hybridizes to the first sequence; and

(c) a third sequence situated between the first and second sequences so as to permit the first and second sequences to hybridize with each other, which third sequence comprises (i) a ribozyme and (ii) a target sequence which is specifically recognized by the ribozyme and is absent in the first and second sequences,

whereby, under hybridizing conditions, the RNA molecule forms a double-stranded RNA complex upon hybridization between the first and second sequences and the target sequence is cleaved by the ribozyme.

The linear dsRNA complex would preferably be formed through expression from an expression vector, and preferably an episomal plasmid or retroviral vector. This could most easily be achieved by constructing a chimeric DNA molecule containing (a) a promoter, operative in the cell, (b) a DNA region capable of being transcribed into an RNA molecule with a nucleotide sequence of at least 20 nucleotides identical with at least part of the nucleotide sequence of the nucleic acid of interest, (c) a DNA region comprising (i) a ribozyme and (ii) a target sequence which is specifically recognized by the ribozyme and is absent in the first and second sequences, (d) a DNA region capable of being transcribed into an antisense sequence wherein the RNA molecule is capable of forming a dsRNA by base pairing between the regions with sense and antisense nucleotide sequence resulting in a intramolecular dsRNA structure, (e) a DNA region encoding a positive selectable marker and (e) a DNA region encoding transcription termination and polyadenylation signals.

In another aspect there is provided a double-stranded RNA complex, which RNA comprises,

(A) a first sequence which, under hybridizing conditions, hybridizes to at least a portion of an mRNA molecule encoded by a gene; and

(B) a second sequence which, under hybridizing conditions, hybridizes to the first sequence; and the second sequence contains at its 3′ end, between the end of the region of complementarity with the first sequence and the polyadenylation signal, a cis-acting hammerhead ribozyme that can cleave within this same region and remove the polyadenylation signal. This embodiment of the invention utilises a ribozyme to cleave the polyadenylation signal of the RNA molecule, thus retaining the RNA molecule and/or dsRNA in the nucleus.

However, the first sequence need not hybridize to at least a portion of an mRNA molecule encoded by the gene. When the RNA molecule or the dsRNA complex is present in the nucleus of a cell, one of the RNA strands need only be complementary to at least part of a gene or a nucleic acid sequence.

The invention also provides a DNA molecule which encodes the linear RNA molecule capable of forming a dsRNA complex.

In case of both the RNA and the DNA molecules, more than one such molecule may be introduced into a cell, whereby the sense and antisense sequences relating to a gene or a nucleic acid sequence are introduced separately and are capable of forming an intermolecular dsRNA complex. In the case of DNA molecules, conveniently these can be introduced on separate vectors and either introduced into a cell simultaneously or sequentially. It will be clear however, that both RNA and DNA molecules and vectors containing them can be introduced into the cell, and into the nucleus, by microinjection, vesicle-mediated transfer or similar techniques well known in the art.

The length of the instant linear RNA molecule must be sufficient to give rise to a dsRNA complex that is at least about 20 nucleotides in length. Although there is no upper limit to the length of the linear RNA molecule or the first and second sequences thereof, in one embodiment, the first and second sequences are each between about 20 and 3000 nucleotides in length. In another embodiment, the first and second sequences are each between about 20 and 25 nucleotides in length. In yet another embodiment, the first and second sequences are each between about 100 and 1000 nucleotides in length. In a further embodiment, the first and second sequences are each between about 200 and 500 nucleotides in length, and preferably each is about 350 nucleotides in length.

The number of ribozymes and target sequences in the third sequence of the instant RNA molecule can be one or a plurality. In the preferred embodiment of the instant RNA molecule, the third sequence comprises a plurality of ribozymes and target sequences cleaved thereby. In one such embodiment, the third sequence comprises two ribozymes and two target sequences cleaved thereby.

In addition, the ribozymes in the third sequence can be any type of ribozymes. In the preferred embodiment, the ribozyme is a hammerhead ribozyme. Within the parameters of this invention, the binding domain lengths (also referred to herein as “arm lengths”) of a ribozyme can be of any permutation, and can be the same or different. Various permutations such as 7+7, 8+8 and 9+9 bases/nucleotides are envisioned. It is well established that the greater the binding domain length, the more tightly it will bind to its complementary mRNA sequence. According, in the preferred embodiment, each binding domain is nine nucleotides in length. A preferred ribozyme is a cis-acting hammerhead ribozyme.

The ribozymes and target sequences within the third sequence of the instant RNA molecule can be situated in a virtually infinite number of ways in order to permit target cleavage and hybridization between the first and second sequences. However, it is preferable that both the ribozymes and their targets reside as close as possible to the junctures with the first and second sequences. For example, in one embodiment, the third sequence comprises the following elements in order: (i) a first target juxtaposed to (e.g., situated within 10 nucleotides of) the first sequence; (ii) a first ribozyme juxtaposed in turn to the first target; (iii) a second ribozyme; and (iv) a second target juxtaposed both to the first sequence and the second ribozyme. In such embodiment, the first ribozyme cleaves the second target and the second ribozyme cleaves the first target, thereby yielding a dsRNA complex without any ribozymes contained within its component RNA strands. Also in this embodiment, the first and second ribozymes may, but need not be, identical, and the first and second targets may, but need not be, identical.

The processing of the dsRNA complex described above using ribozymes can also be achieved by providing in the third sequence an intron and appropriate splice donor/acceptor sites that upon transcription the cell machinery will splice out the intron leaving dsRNA.

The ribozyme may also be contained within the second sequence as described earlier and in this construct can cleave the polyadenylation signal, and assist in the retention of the RNA molecules and the dsRNA complex in the nucleus. Anyone skilled in the art would realize that the cis-acting ribozyme approach described is but one of a number of different ways to retain RNA within the nucleus. For example, the inclusion of a recognition signal for a nuclear based RNA binding protein may also be used. In addition any known nuclear RNA localization sequences may be included to achieve nuclear retention of the RNA.

In the instant RNA molecule, the third sequence can contain any additional sequences intended to facilitate the formation and/or monitoring of dsRNA formation. Such sequences include, without limitation, exogenous genes such as those conferring drug resistance, or markers which facilitate detection of gene suppression or loss of the intervening sequence (such as negative selectable markers including, but not restricted to, herpes simplex thymidine kinase, E. coli cytosine deaminase, etc). The present invention also provides a first composition for use in inhibiting the expression of a gene in a eukaryotic cell comprising

(a) a RNA molecule encoding HIV Tat protein; and

(b) a linear RNA molecule for forming a double-stranded RNA complex, which RNA molecule comprises

(i) a first sequence which, under hybridizing conditions, hybridizes to at least a portion of an mRNA molecule encoded by the gene;

(ii) a second sequence which, under hybridizing conditions, hybridizes to the first sequence; and

(iii) a third sequence situated between the first and second sequences so as to permit the first and second sequences to hybridize with each other, whereby, under hybridizing conditions, the RNA molecule forms a double-stranded RNA complex upon hybridization between the first and second sequences.

This invention further provides a DNA molecule encoding the RNA molecules of the first composition. In the preferred embodiment, the DNA molecule is operably situated within an expression vector.

This invention further provides a second composition for use in inhibiting the expression of a gene in a eukaryotic cell comprising

(a) a DNA molecule encoding a RNA portion that enhances the ability of dsRNA to alter transcription from an mRNA molecule encoded by a gene; and

(b) a DNA molecule encoding a linear RNA molecule for forming a double-stranded RNA complex, which RNA molecule comprises

(i) a first sequence which, under hybridizing conditions, hybridizes to at least a portion of an mRNA molecule encoded by the gene;

(ii) a second sequence which, under hybridizing conditions, hybridizes to the first sequence; and

(iii) a third sequence situated between the first and second sequences so as to permit the first and second sequences to hybridize with each other, whereby, under hybridizing conditions, the RNA molecule forms a double-stranded RNA complex upon hybridization between the first and second sequences. In one embodiment the enhancing portion is a portion encoding the HIV Tat protein.

In the preferred embodiment, each DNA molecule is operably situated within an expression vector.

In one embodiment of the compositions described above, the third sequence of the linear RNA molecule comprises (i) a ribozyme and (ii) a target sequence specifically recognized by the ribozyme and absent in the first and second sequences, whereby the complex-forming portion forms a double-stranded RNA complex upon hybridization between the first and second sequences and cleavage of the target sequence by the ribozyme. In a preferred embodiment, the ribozyme is a hammerhead ribozyme.

The composition described above may also use one or more DNA molecules encoding the RNA molecules capable of forming the dsRNA.

This invention further provides (i) an expression vector comprising the instant RNA molecule, (ii) a DNA molecule encoding the instant RNA molecule, and (iii) an expression vector comprising the instant DNA molecule. It will be clear that more than one RNA and DNA molecule can be used in the generation of the dsRNA complex and each such molecule can be comprised in a separate vector. The RNA molecules, the DNA molecules and the vectors comprising them can be introduced into a cell simultaneously or sequentially. Expression vectors (e.g., retroviral expression vectors such as LNL6, and adenoviral expression vectors) and their uses are well known in the art (Sambrook et al., 1989), and these vectors can be integrating or non-integrating vectors.

This invention further provides a cell comprising the instant RNA molecule and/or the DNA molecule encoding same, as well as a cell comprising the instant expression vector comprising the instant RNA molecule and/or the DNA molecule encoding same. In the preferred embodiment, the cell is a eukaryotic cell. Eukaryotic cells include, without limitation, Hela cells, fibroblasts, astrocytes, neurons, NB41 cells, T-lymphocytes, monocytes, CD34⁺ stem cells and SupT-1 cells. It also includes differentiated and undifferentiated somatic cells and neoplastic cells.

This invention provides methods of forming a double-stranded RNA complex in a cell which comprises introducing into the cell the instant RNA molecule, thereby permitting the molecule to form a double-stranded RNA complex. Also provided by this invention is the dsRNA complex formed by this method. The RNA molecule may be introduced directly into a cell or may be introduced by way of a DNA molecule encoding the RNA molecule. Both RNA and DNA molecules may be introduced with the aid of a vector.

The invention further provides a method of suppressing expression of a specified gene or a specified nucleic acid sequence in a eukaryotic cell comprising introducing into said cell one or more RNA molecules or one or more DNA molecules encoding the one or more RNA molecules, wherein said one or more RNA molecules comprises first and second sequences corresponding to sense and antisense sequences with respect to the specified gene or the specified nucleic acid sequence and wherein said DNA molecule comprises sequences which encode first and second RNA molecules corresponding to sense and antisense sequences with respect to the specified gene or the specified nucleic acid sequence.

In one embodiment the method comprises introducing into the cell (a) a double-stranded RNA complex, at least one of whose strands hybridizes to at least a portion of an mRNA molecule encoded by the gene under hybridizing conditions; and (b) HIV Tat protein. In another embodiment the method comprises introducing into the cell the instant DNA expression vector. In yet another embodiment the method comprises introducing into the cell the instant DNA expression vector-containing composition. In still further embodiment the method comprises introducing into a cell a pair of DNA molecules, each of which encodes one strand of the dsRNA complex. Each DNA molecule may be introduced by way of a separate vector.

Genes whose expression can be inhibited by the instant method include, without limitation, genes relating to cancer, rheumatoid arthritis and viruses. Cancer-related genes include oncogenes (e.g., K-ras, c-myc, bcr/abl, c-myb, c-fms, c-fos and cerb-B), growth factor genes (e.g., genes encoding epidermal growth factor and its receptor, fibroblast growth factor-binding protein), matrix metalloproteinase genes (e.g., the gene encoding MMP-9), adhesion-molecule genes (e.g., the gene encoding VLA-6 integrin), tumor suppressor genes (e.g., bcl-2 and bcl-Xl), angiogenesis genes, and metastatic genes. Rheumatoid arthritis-related genes include, for example, genes encoding stromelysin and tumor necrosis factor. Viral genes include human papilloma virus genes (related, for example, to cervical cancer), hepatitis B and C genes, and cytomegalovirus genes (related, for example, to retinitis).

In one embodiment of the instant method, the cell is HIV-infected and the gene is an HIV gene. HIV genes include, without limitation, tat, nef, rev, ma (matrix), ca (capsid), nc (nucleocapsid),p6, vpu, pr (protease), vif, su (gp120), tm (gp41), vpr, rt (reverse transcriptase) and in (integrase). In the preferred embodiment, the HIV gene is tat.

This invention further provides a method of inhibiting the expression of an HIV gene in an HIV-infected eukaryotic cell, which comprises introducing into the cell a double-stranded RNA complex comprising an RNA sequence that hybridizes to at least a portion of the mRNA encoded by the HIV gene whose expression is to be inhibited. HIV genes include, without limitation, tat, nefs rev, ma, ca, nc, p6, vpu, pr, vif, su, tm, vpr, rt and in. In the preferred embodiment, the HIV gene is tat.

In another method of this invention relates to a method for localizing a dsRNA molecule in the nucleus of a cell. This method comprises introducing one or more RNA molecules into a cell or DNA encoding one or more RNA molecules such that the RNA molecules form a dsRNA complex in a cell where the RNA molecule includes a first portion that hybridizes to at least a portion of a mRNA molecule encoded by a gene, and a second portion wherein at least part of the second portion is capable of hybridizing to the first portion and wherein the second portion comprises a polyadenylation signal and a ribozyme positioned between the part of the second portion capable of hybridizing to the first portion and the polyadenylation signal wherein the ribozyme is capable of removing the polyadenylation signal thereby retaining the RNA in the nucleus.

This invention further provides pharmaceutical compositions for inhibiting the expression of a gene in the cells of a subject. One such composition comprises (a) a double-stranded RNA complex, at least one of whose strands hybridizes to at least a portion of an mRNA molecule encoded by the gene under hybridizing conditions; (b) HIV Tat protein; and (c) a pharmaceutically acceptable carrier. Another comprises (a) the instant DNA expression vector, and (b) a pharmaceutically acceptable carrier. Yet another comprises (a) the instant DNA expression vector-containing composition, and (b) a pharmaceutically acceptable carrier. Further compositions comprise a pair of RNA or DNA molecules capable of generating a dsRNA complex, vectors comprising such RNA and DNA molecules, and the instant dsRNA complex, in combination with a pharmaceutically acceptable carrier.

This invention also provides a method of treating a subject having a disorder ameliorated by inhibiting the expression of a known gene in the subject's cells, comprising administering to the subject a therapeutically effective amount of the instant pharmaceutical compositions wherein, under hybridizing conditions, the first sequence hybridizes to at least a portion of an mRNA encoded by the gene whose expression is to be inhibited.

This invention also provides a method of inhibiting in a subject the onset of a disorder ameliorated by inhibiting the expression of a known gene in the subject's cells, comprising administering to the subject a prophylactically effective amount of the instant pharmaceutical composition wherein, under hybridizing conditions, the first sequence hybridizes to at least a portion of an mRNA encoded by the gene whose expression is to be inhibited.

Known genes whose expression can be inhibited by the instant methods include, without limitation, genes relating to cancer, rheumatoid arthritis and viruses. Cancer-related genes include oncogenes (e.g., K-ras, c-myc, bcr/abl, c-myb, c-fms, c-fos and cerb-B), growth factor genes (e.g., genes encoding epidermal growth factor and its receptor, and fibroblast growth factor-binding protein), matrix metalloproteinase genes (e.g., the gene encoding MMP-9), adhesion-molecule genes (e.g., the gene encoding VLA-6 integrin), and tumor suppressor genes (e.g., bc/-2 and bcl-Xl). Rheumatoid arthritis-related genes include, for example, genes encoding stromelysin and tumor necrosis factor. Viral genes include human papilloma virus genes (related, for example, to cervical cancer), hepatitis B and C genes, and cytomegalovirus genes (related, for example, to retinitis). In one embodiment of the instant method, the cell is HIV-infected and the gene is an HIV gene. HIV genes include, without limitation, tat, nef, rev, ma, ca, nc, p⁶, vpu, pr, vif, su, tm, vpr, rt and in. In the preferred embodiment, the HIV gene is tat.

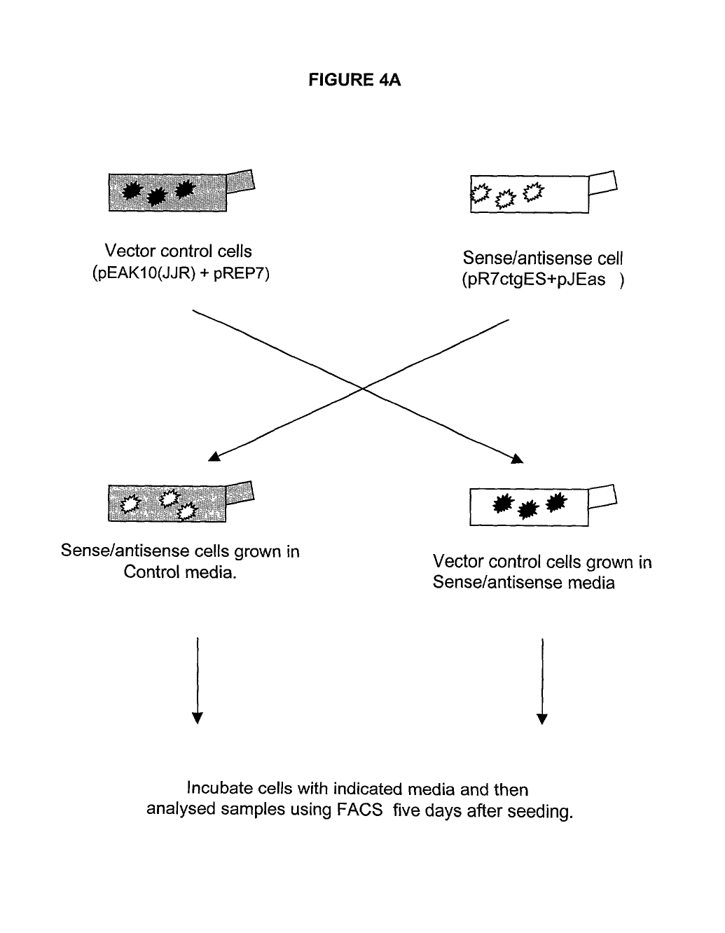

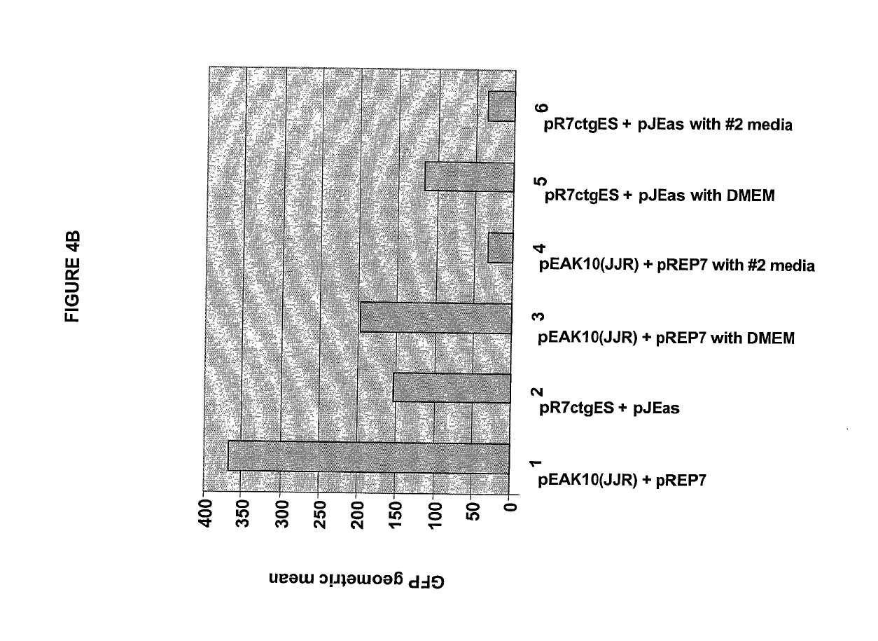

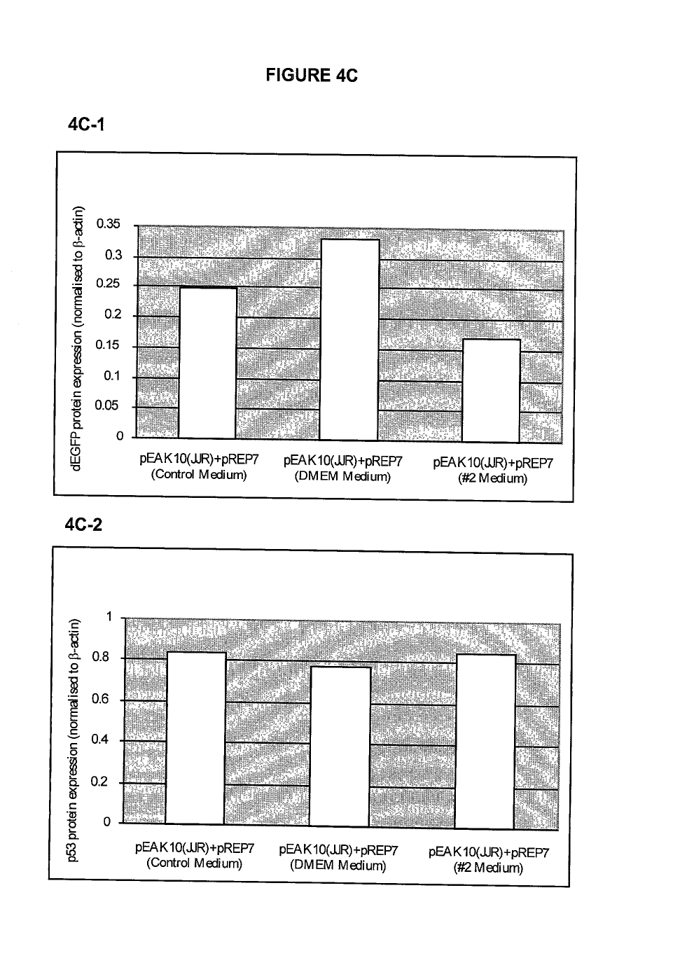

The invention further provides a method for modulating expression (preferably suppressing or inhibiting expression of a gene) of a nucleic acid sequence in a cell comprising exposing the cell to culture medium that has been removed from cells that were grown in culture and contained within them dsRNA complexes that comprised a first portion that hybridizes to at least part of a mRNA molecule encoded by a gene and a second portion wherein at least part of the second portion is capable of hybridizing to the first portion. This embodiment is further described as it relates to FIG. 4.

Determining a therapeutically or prophylactically effective amount of the instant pharmaceutical composition can be done based on animal data using routine computational methods. In one embodiment, the therapeutically or prophylactically effective amount contains between about 0.1 mg and about 1 g of the instant nucleic acid molecules. In another embodiment, the effective amount contains between about 1 mg and about 100 mg of the nucleic acid molecules. In a further embodiment, the effective amount contains between about 10 mg and about 50 mg of the nucleic acid molecules, and preferably about 25 mg thereof.

In this invention, administering the instant pharmaceutical composition can be effected or performed using any of the various methods and delivery systems known to those skilled in the art. The administering can be performed, for example, intravenously, orally, via implant, transmucosally, transdermally, intramuscularly, and subcutaneously. In addition, the instant pharmaceutical compositions ideally contain one or more routinely used pharmaceutically acceptable carriers. Such carriers are well known to those skilled in the art. The following delivery systems, which employ a number of routinely used carriers, are only representative of the many embodiments envisioned for administering the instant composition.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Examples of liposomes which can be used in this invention include the following: (1) CellFectin, 1:1.5 (M/M) liposome formulation ofthe cationic lipid N,N^{I},N^{II},N^{III}-tetramethyl-N,N^{I},N^{II},N^{III}-tetrapalmityl-spennine and dioleoyl phosphatidylethanolamine (DOPE)(GIBCO BRL); (2) Cytofectin GSV, 2:1 (M/M) liposome formulation of a cationic lipid and DOPE (Glen Research); (3) DOTAP (N-[1-(2,3-dioleoyloxy)-N,N,N-trimethyl-ammoniummnethylsulfate) (Boehringer Manheim); and (4) Lipofectamine, 3:1 (M/M) liposome formulation of the polycationic lipid DOSPA and the neutral lipid DOPE (GIBCO BRL).

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

Numerous experimental methods are relevant to this invention or experiments leading thereto, which are within routine skill in the art. These include: methods for isolating nucleic acid molecules, including, for example, phenol chloroform extraction, quick lysis and capture on columns [Kramvis et al., 1996; Sambrook et al., 1989, U.S. Pat. No. 5,582,988 and Yong et al. (1995)]; methods of detecting and quantitating nucleic acid molecules; methods of detecting and quantitating catalytic nucleic acid activity; methods of amplifying a nucleic acid sequence including, for example, PCR, SDA and TMA (also known as (SSR))[ Chehab et al., 1987; Fahy et al., 1991; Jonas, V., et al., 1993; Saiki et al., 1985; U.S. Pat. Nos. 4,683,202; 4,683,195; 4,000,159; 4,965,188; 5,176,995; Walder et al., 1993; Walker et al., 1992]; and methods of determining whether a catalytic nucleic acid molecule cleaves an amplified nucleic acid segment including, by way of example, polyacrylamide gel electrophoresis and fluorescence resonance energy transfer (FRET) [Cuenoud and Szostak, 1995; and PCT International Publication No. WO 94/29481].

In a broad description of an ex vivo therapeutic application of double-stranded RNA, the constructs encoding the gene therapeutic are included in a retroviral-based replication incompetent virus. The therapeutic would then be applied by methods well known in the art to stem cells, ex vivo. The stem cells may be isolated from patients by methods well known in the art. The genetically modified stem cells are then transferred back into the patient by infusion where the influence on disease would be exerted by the genetically modified cells expressing double-stranded RNA.

In a specific example for HIV therapy, the gene transfer product comprises a Moloney Murine Leukemia Virus (MoMLV)-based, replication incompetent retroviral vector (LNL6) containing the H5 and ASH5 sequences that upon transcription yields a dsRNA molecule homologous to nucleotides between 530-1089 of the HIV genome (HXB2). The examples given in FIG. 9 depict various compositions of vectors that could be used. The treatment includes the mobilisation of hematopoietic progenitor cells (CD34+ cells) by Granulocyte-Colony Stimulating Factor (G-CSF), from the bone marrow and collection by apheresis. CD34+ cells can be enriched and cultured ex vivo by methods well known in the art. The CD34+ cells are transduced with replication incompetent retrovirus encoding dsRNA before being reinfused back into the patient. The dsRNA containing CD34+ cells then migrate to the bone marrow and in time contribute to the peripheral lymphocyte population. The dsRNA offers protection from HIV infection and a reduced amount of viral production within infected cells.

The present invention also provides methods for determining function of a gene or a nucleic acid and methods for determining function of a protein by suppressing expression of a gene or a nucleic acid.

In another aspect the invention provides a method of modulating expression of a gene or a nucleic acid sequence in mammalian cells including exposing said cells to medium in which mammalian cell of the present invention has been grown. In this embodiment, medium exposed to mammalian cells expressing chimeric DNA molecules encoding the dsRNA complexes described within the present invention may be used to modify the expression of the specific target gene within mammalian cells that do not harbour the chimeric DNA molecules. To those skilled in the art it is obvious that the medium contains a specific silencing signal that can be transferred using the medium described in Example 3. This signal can be derived as described in Example 3 or it can be derived from other cell types (such as drosophila or plant cells) that are capable of forming this secretory silencing signal.

This invention will be better understood by reference to the Experimental Details which follow, but those skilled in the art will readily appreciate that the specific experiments detailed are only illustrative of the invention as described more fully in the claims which follow thereafter. Additionally, throughout this application, various publications are cited. The disclosure of these publications is hereby incorporated by reference into this application to describe more fully the state of the art to which this invention pertains.

### EXAMPLE 1

### Materials and Methods to Exemplify the Invention

Construction of Episomal Expression Vectors

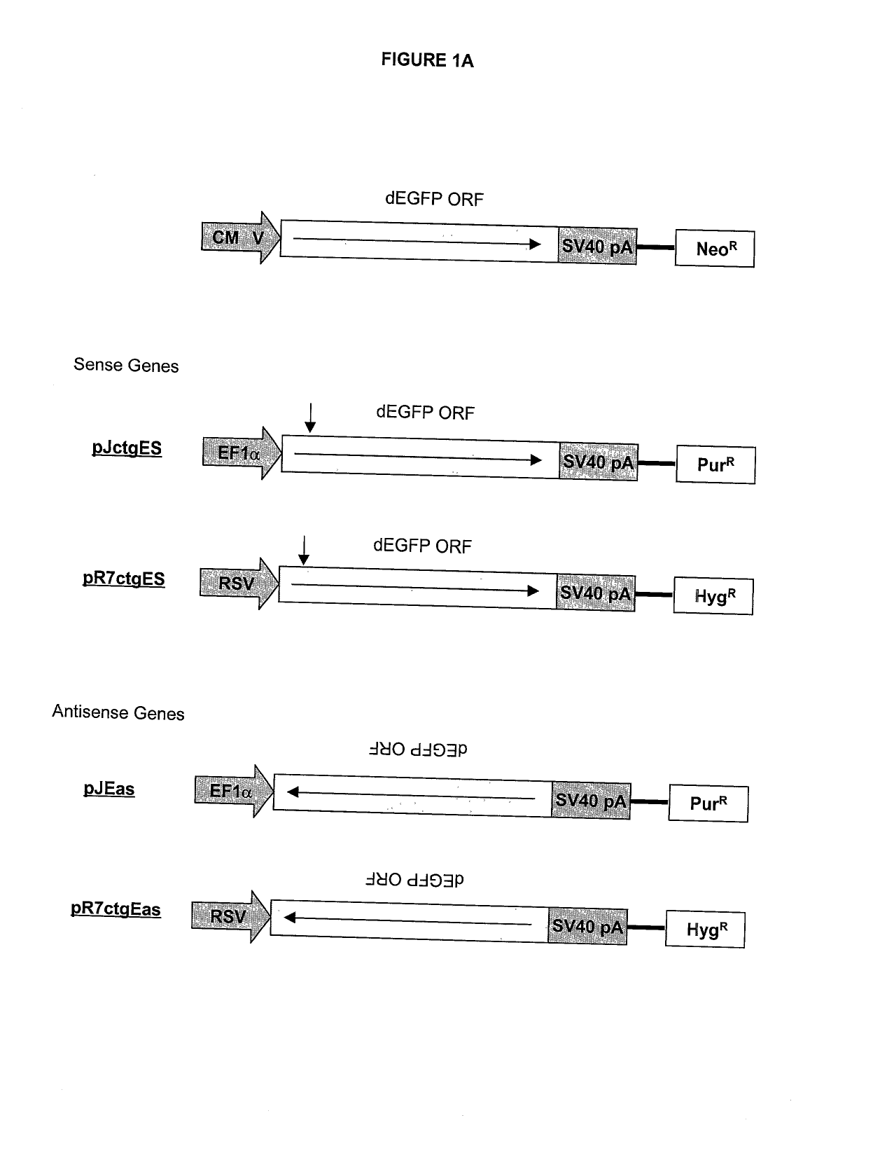

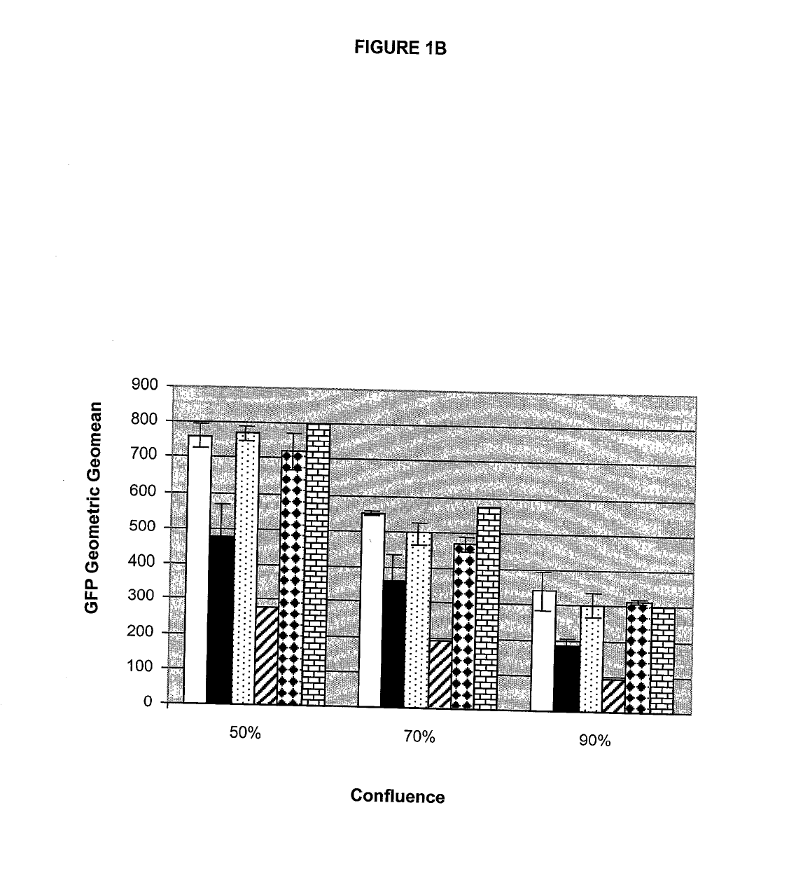

Standard gene cloning methods were used to construct expression plasmids used in the present study (Sambrook et al., 1989). The plasmids used in the co-transfection experiments were based in the core episomal plasmids pREP7 (Invitrogen) or pEAK10 (Edge Biosystems). These plasmids are maintained within the nucleus and do not generally integrate into the genomic DNA. The sequences required for episomal plasmid maintenance are the Epstein Barr virus OriP and EBNA1 regions, known in the art. The portion of the dEGFP target gene used to construct the sense and antisense dEGFP-expressing plasmids in pREP7 spanned positions 666 to 1749 in reference to the pd4EGFP-N1 (Clontech) sequence map. This region was PCR-amplified using pd4EGFP-N1 as a template and the following primers: 5′ TGA GGA TTC ACC GGT CGC CAC CCT GGT GAG CAA G 3′ (SEQ ID NO:1) and 5′ TGA GGA TTC ACA AAC CAC AAC TAG AAT GCA GTG 3′ (SEQ ID NO:2) (The base change indicated by C was introduced to eliminate the ATG start codon and ensure that sense dEGFP RNA was not translated). The 1080 bp PCR product was digested with BamHI and subcloned into the unique BamHI site in pREP7 downstream of the RSV LTR promoter in the sense and antisense orientations to produce pR7ctgES and pR7ctgEaS, respectively. The dEGFP insert in plasmid pJEAs was obtained by PCR amplifying the entire transcription unit of the dEGFP gene spanning positions 583 to 1749 (in reference to the pd4EGFP-N1 sequence map) using the following PCR primers: 5′ TCA GAT CCG CTA GCG CTA CCG GAC 3′ (SEQ ID NO:3) and 5′ ACA AAC CAC AAC TAG AAT GCA GTG 3′ (SEQ ID NO:4). This fragment was ligated to BamHI adaptors created by annealing the following single stranded oligonucleotides: 5′ TCT CTA GGG ATC CTC AGT CAG TCA GGA TG 3′ (SEQ IDNO:5) and 5° CAT CCT GAC TGA CTG AGG ATC CCT AGA GAA TA 3′ (SEQ ID NO:6). The adaptor-ligated fragment was then digested with BamHI and ligated into the unique BglII site in pEAK10 (JJR) in the antisense orientation relative to the mammalian protein elongation factor 1α promoter to produce pJEAs. For construction of the plasmid pJctgES, the region of the dEGFP gene in pd4EGFP-N1 spanning positions 666 to 1749 was PCR-amplified using the forward primer 5′ TGA AGA TCT ACC GGT CGC CAC CCT GGT GAG CAA G 3′ (SEQ ID NO:7) and the reverse primer 5′ TGA GAA TTC ACA AAC CAC AACTAG AAT GCA GTG 3′ (SEQ ID NO:8). The BglII-EcoRI digested PCR product was directionally cloned in the sense direction downstream of the elongation factor 1α. promoter of pEAK10(JJR) to produce pJctgES. The sense and antisense dEGFP genes contained on pR7ctgES, pR7ctgEaS, pJEAs, and pJctgES are indicated in FIG. 1A.

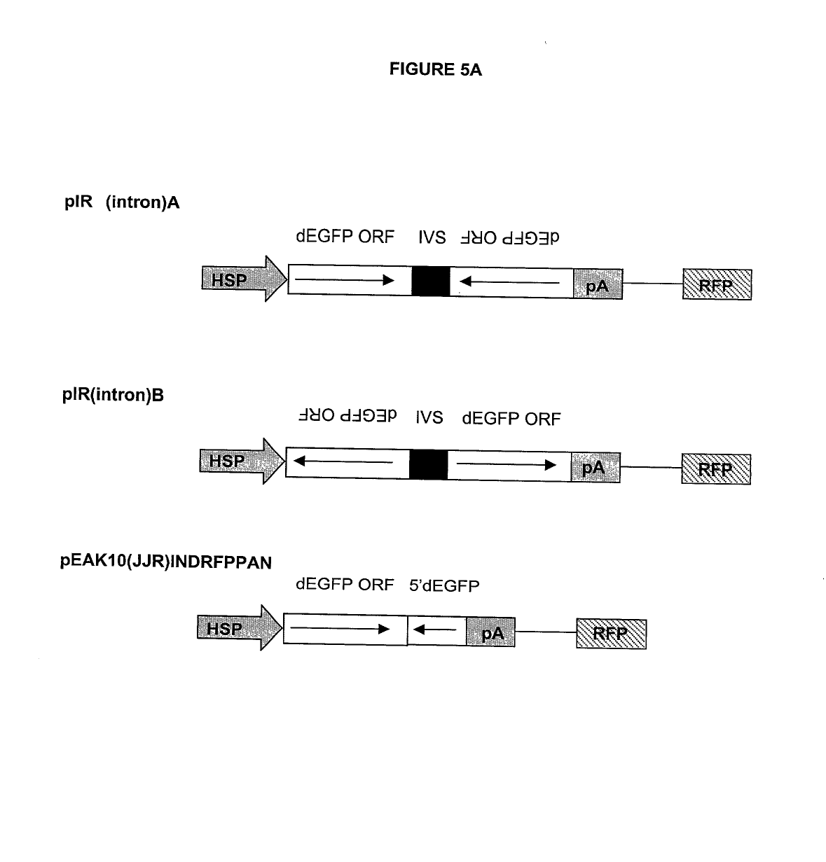

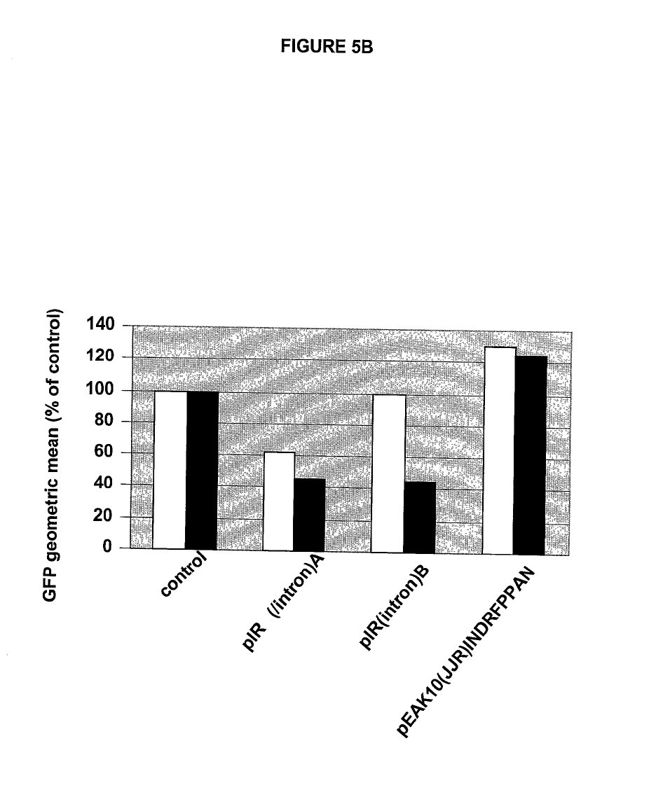

The expression cassettes resident on the inverted repeat plasmids are summarised in FIG. 5A. The core plasmid was based on pEAK10 (Edge Biosystems). The elongation factor 1α promoter on pEAK10(JJR) was replaced by the heat shock minimal promoter (containing ecdysone/glucocorticoid response elements), the latter of which is conditionally induced in the EcR293 cell line upon addition of the analog ponasterone A. This was accomplished by PCR amplifying the heat shock promoter region using the forward primer 5′ TGA ACT AGT TCT CGG CCG CAT ATT AAG TGC 3′ (SEQ ID NO:9) and the reverse primer 5′ TGA AAG CTT AAG TTT AAA CGC TAG 3′ (SEQ ID NO:10) and pIND (Invitrogen) as a template. The PCR product was digested with SpeI and HindIII and subcloned directionally into pEAK10(JJR) in place of the elongation factor 1α promoter to produce the plasmid pEAK10(JJR)IND. This vector was further modified to include the RFP gene derived from pDsRed1-N1 (Clontech). This involved digesting pDsRed1-N1 with NheI and AgeI, end-illing, and self-ligating to eliminate the multiple cloning site. The RFP cassette was then PCR-amplified from the modified pDsRed1-N1 using the following PCR primers: 5′ GCGC ACT AGT CGT ATT ACC GCC ATG CAT TAG 3′ (SEQ ID NO: 11) and 5′ GCGC ACT AGT ACG CCT TAA GAT ACA TTG ATG 3′ (SEQ ID NO:12). The SpeI-digested product was cloned into SpeI-linearised pEAK10(JJR)IND to produce pEAK10(JJR)INDRFP. This latter vector was the core plasmid used to construct the inverted repeat plasmids.

For construction of the chimeric gene contained on plasmid pEAK10(JJR)INDRFPPAN, the region of the dEGFP gene spanning position 666 to 1527 (in relation to the pd4EGFP-N1 map) was PCR-amplified from pJctgES using the forward primer 5′ GCGC AGA TCT ACC GGT CGC CAC CCT GGT GAG 3′ (SEQ ID NO:13) and the reverse primer 5′ GCGC GAA TTC CAT CTA CAC ATT GAT CCT AG 3′ (SEQ ID NO: 14). This 862 bp fragment was digested with BglII and EcoRI and directionally cloned in the sense orientation downstream of the conditional heat shock promoter in pEAK10(JJR)INDRFP. To complete construction of the chimeric gene on pEAK10(JJR)INDRFPPAN, a 350 bp region from the 5′ end of the dEGFP (corresponding to positions 666 to 1020 of the pd4EGFP-N1 vector) was PCR-amplified using the primers 5′ TGA GAA TTC AGA TCT ACC GGT CGC CAC CCT GGT TGA GCA AG 3′ (SEQ ID NO:15) and 5′ TGA GAA TTC CTT CAC CTC GGC GCG GGT CTT GTA G 3′ (SEQ ID NO: 16), and cloned as an EcoRI fragment in the antisense orientation downstream of the 862 bp dEGFP fragment to form the inverted repeat cassette.

To construct the intron-containing chimeric genes contained on plasmids pIR(intron)A and pIR(intron)B, a three step cloning protocol was followed. In the first step, the 862 bp region of the dEGFP gene spanning position 666 to 1527 (in relation to the pd4EGFP-N1 map) was PCR-amplified from pJctgES using the forward primer 5′ GCGC AGA TCT ACC GGT CGC CAC CCT GGT GAG 3′ (SEQ ID NO:17) and the reverse primer 5′ GCGC AGA TCT CAT CTA CAC ATT GAT CCT AG 3′ (SEQ ID NO:18), and cloned as a BglII fragment in both orientations downstream of the conditional heat shock promoter in pEAK10(JJR)INDRFP. In the second step, the 296 bp synthetic intervening sequence spanning positions 974 to 1269 of the vector pIRES-Neo (Clontech) was PCR-amplified using the primers 5′ GCGC GGT ACC GAA TTA ATT CGC TGT CTG CGA 3′ (SEQ ID NO:19) and 5′ GCGC GGT ACC CGA CCT GCA CTT GGA CCT GG 3′ (SEQ ID NO:20), and cloned as a KpnI fragment in the sense direction downstream of the dEGFP fragment cloned in the first step. The final step in the construction process involved PCR amplification of the 862 bp region of the dEGFP gene spanning position 666 to 1527 (in relation to the pd4EGFP-N1 map) from pJctgES using PCR primers that introduced XbaI and EcoRI sites to the amplified fragment. These fragments were cloned directionally downstream of the intron sequences to produce the inverted repeat genes on plasmids pIR(intron)A and pIR(intron)B, as summarised in FIG. 5A.

The construction of the plasmids to examine the utility of using a cis-acting hammerhead ribozyme to restrict transport of RNAs from the nucleus to the cytoplasm was initiated by PCR-amplifying the humanised GFP open reading frame from pGREENLANTERN (Life Technologies) using the 5′ primer 5′ TGA AAG CTT GCC GCC ACC ATG AGC AAG GGC GAG 3′ (SEQ ID NO:21) and the 3′ primer 5′ TGA AAG CTT TCA CTT GTA CAG CTC GTC CAT GCC 3′ (SEQ ID NO:22). Cis-actinb ribozymes are known in the art including those descriptions of Eckner, et al. (EMBO 10(11): 3513-3522, 1991) and Liu, et al. (Proc. Natl. Acad. Sci. USA 91:4258-4262, 1994). This DNA was then cloned as a HindIII fragment in the sense direction under control of the elongation factor loc promoter on pEAK10(JJR) to produce pEAK10gfps. The cis-acting ribozyme-encoding DNA was obtained by sythesising and annealing the following complementary oligonucleotides: 5′ GAA TTC AAT TCG GCC CTT ATC AGG GCC ATG CAT GTC GCG GCC GCC TCC GCG GCC GCC TGA TGA GTC CGT GAG GAC GAA ACA TGC ATA GGG CCC TGAT 3′ (SEQ ID NO:23) and 5′ ATC GGG CCC TAT GCA TGT TTC GTC CTC ACG GAC TCA TCA GGC GGC CGC GGA GGC GGC CGC GAC ATG CAT GGC CCT GAT AAG GGC CGA ATT G 3′ (SEQ ID NO:24). This DNA was digested with EcoRI and ligated directionally into the pEAK10gfs plasmid following digestion with EcoRI and EcoRV. The end result was the plasmid pEAK10gfps+RBZ. For each of the plasmids pEAK10gfps and pEAK10gfps+RBZ, derivatives were constructed in which the SV40 polyadenylation signal downstream of the GFP ORF in pEAK10gfps and the cis-acting ribozyme sequence in pEAK10gfps+RBZ was deleted. This produced plasmids pEAK10gfps-pA and pEAK10gfps+RBZ-pA, respectively.

Construction of dEGFP-expressing Cell Line

The derivative cell line expressing the dEGFP target gene was constructed by electroporating EcR293 cells (Invitrogen) with the plasmid pd4EGFP-N1 (Clontech) that had been linearised with AflII. The transfected cell population was selected in the presence of 500 μg/ml G418 and Neo^{R} clones expanded and screened for dEGFP expression using fluorescence-activated cell sorting (FACs) analyses. The cell line expressing dEGFP under control of the CMV immediate early promoter was shown to contain a single copy of the dEGFP expression cassette.

Cell Culture and Methods

EcR293 human embryonic kidney cells (Invitrogen) and their derivatives were maintained in DMEM containing 10% fetal calf serum and supplemented with glutamine, streptomycin and penicillin. This cell line expresses a heterodimer of the ecdysone receptor (VgEcR) and the retinoid X receptor (RXR) that binds a hybrid ecdysone response element in the presence of the analog of ecdysone, ponasterone A (No et al., 1996; Saez et al., 2000). FACs analysis for GFP or RFP expression was performed on the Becton Dickinson FACSORT. Total RNA was extracted from cells using the TRIZol Reagent (Life Technologies, Inc.) according to the manufacturer's instructions. Northern hybridisation method for target mRNA detection was performed according to Sambrook et al (1989).

To select for cells co-transfected with two different episomal plasmids, a total of 2.5×10⁶ dEGFP-expressing cells were electroporated with 2.5 μg of each of the plasmids. At 48 hours after transfection, cells were exposed to 0.7 μg/ml of puromycin (to select for pEAK10-based plasmids) and 100 μg/ml hygromycin (to select for pREP7-based plasmids). At 28 days after double selection, cells were then exposed to triple selection by including 500 μg/ml of G418. At five weeks post-electroporation, each of the selected populations was characterised for dEGFP-mediated cell fluorescence, dEGFP protein level and steady-state level of dEGFP mRNA.

To test the inverted repeat plasmids in a transient assay, a total of 5 μg of each of these plasmids and the control vector was independently electroporated into 1×10⁶ dEGFP-expressing cells. At 48 hours post-transfection, each cell population was treated with either 10 μM ponasterone A (induction conditions) or vehicle alone (no induction). At 24 hours after this treatment, cells were harvested and analysed for dEGFP-mediated cell fluorescence. This involved gating for RFP positive cells (transfected cells only) and determining the dEGFP fluorescence profile within this sub-population.

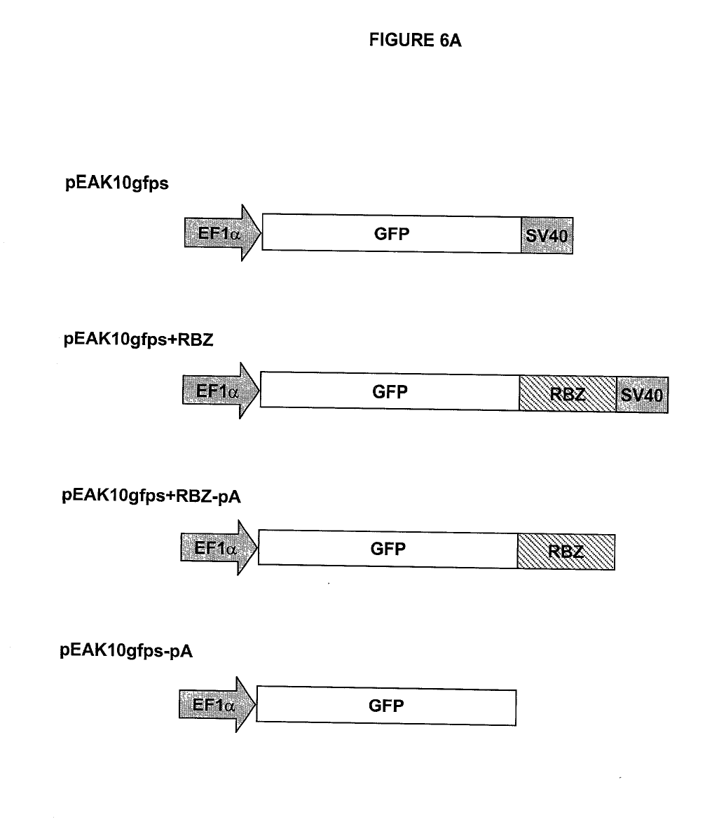

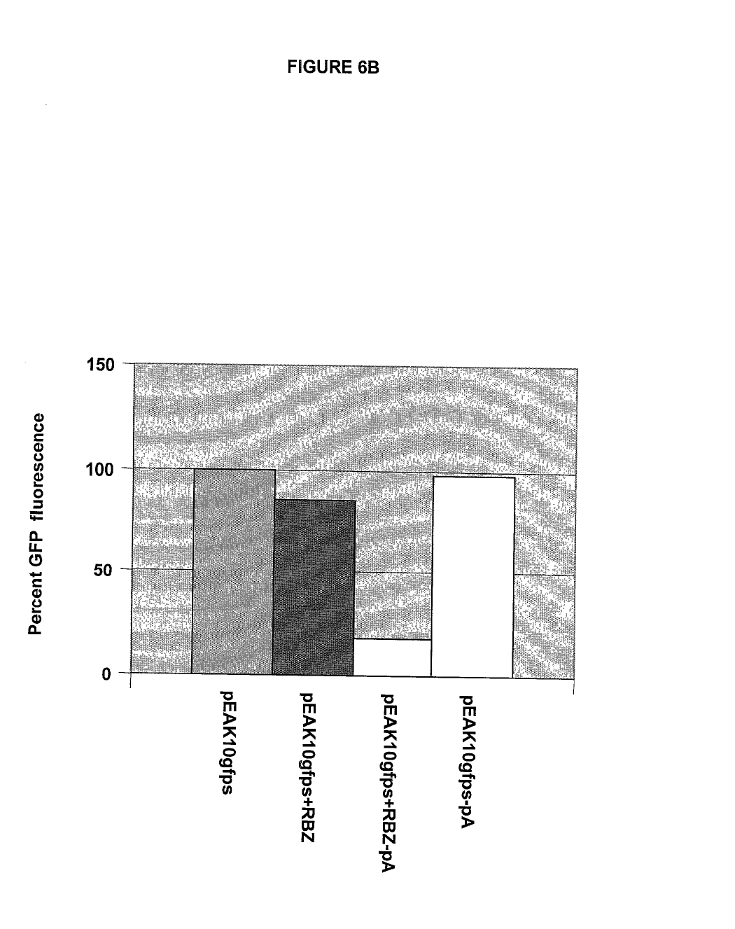

To examine the effect of the cis-acting hammerhead ribozyme on retention of RNA within the nucleus, each of the constructs indicated in FIG. 6A was introduced into dEGFP-expressing cells by electroporation. At 48 hours after transfection, cells containing the episomal plasmids were selected by adding 1 mg/ml puromycin. Following three weeks of selection, puromycin resistant cells were harvested and assayed for dEGFP-mediated cell fluorescence.

Western Blotting Procedures

Cell lysates were prepared using RIPA buffer supplemented with protease inhibitors aprotonin (1 μg/ml), leupeptin (10 μg/ml) and DMSF (100 μg/ml). A total of 60μg of total protein was loaded onto pre-cast 10% agarose Tris-HCL gels (BioRad). Proteins were separated by electrophoresis at 200 volts for 1 hour and transferred to PVDF membrane (Millipore) at 80 volts for 60-90 minutes. This membrane was probed with either GFP mouse polyclonal (Clontech), PKR rabbit polyclonal (SantaCruz) or β-actin mouse monoclonal (SantaCruz) antibodies. Secondary antibody detection was performed using either the goat anti-mouse (horseradish peroxidase (HRP)-linked) or the goat anti-rabbit HRP (SantaCruz), followed by visualisation using the luminol/enhancer chemiluminescent substrate (Amersham).

Media Transfer Experiments

To examine the effect of the culture medium on dEGFP-mediated cell fluorescence, control cells and cells co-expressing antisense and sense dEGFP RNA were each seeded in three media types: control cell conditioned medium, sense/antisense cell conditioned medium and DMEM medium. After two and five days in each of these media, both control cells and cells co-expressing antisense and sense dEGFP RNA were assayed for cell fluorescence using FACs.

Reverse Transcriptase PCR (RT-PCR)

To detect expression of the antisense and sense dEGFP RNAs, standard RT-PCR reactions were performed. The reaction conditions included 500 ng of total RNA, 25 nM of the reverse primer, 5 nM of the forward primer, 6 units of Moloney Murine Leukemia Virus (M-MuLV) RT (New England BioLabs, USA), 1×PCR Gold buffer (15 mM Tris_HCl, pH 8.0, 50 mM KCl; Perkin Elmer, USA), 4 mM MgCl₂, 1.8 units of Taq Gold (Perkin Elmer, USA), 10 mM dNTPs, and 10 units of RNasin (Promega, USA). For the sense dEGFP RNA, the following primers were used: forward primer 5′ GCAATTGAACCGGTGCCTAGA 3′ (SEQ ID NO:25) and reverse primer 5′ GAACTTGTGGCCGTTTAC 3′ (SEQ ID NO:26). For the antisense dEGFP RNA, the following primers were used: forward primer 5′ CGCAGATCCTGAGCTTGTATG 3′ (SEQ ID NO:27) and reverse primer 5′ CACTGCATTCTAGTTGTG 3′ (SEQ ID NO:28). In each case the cycling conditions were performed in two steps. In the reverse transcription step, the reactions were incubated at 50° C. for 60 minutes followed by 95° C. for 10 minutes to inactivate the Taq antibody. In the PCR step, the cycling conditions were 94° C. for 3 minutes, followed by 30 cycles of 94° C. for 1 minute, 65° C. for 1 minute and 72° C. for 2 minutes. Finally, the entire reaction was incubated at 72° C. for 10 minutes. Using these reaction conditions, both the sense dEGFP and antisense dEGFP RNA were detected as 260 bp and 680 bp RT-PCR products, respectively. Neither of these bands were observed in the absence of the M-MuLV RT. DNA sequencing of these RT-PCR products indicated that each was derived from the relevant dEGFP RNA.

To detect expression of the human homologue of Dicer, the RT-PCR conditions described above were used with the following Dicer-specific primers: forward primer 5′ TTAACCAGCTGTGGGGAGAGGGCTG 3′ (SEQ ID NO:29) and reverse primer 5′ AGCCAGCGATGCAAAGATGGTGTTG 3′ (SEQ ID NO:30). Amplification from total RNA produced the expected 579 base pair RT-PCR product.

### EXAMPLE 2

### The Effect of Sense RNA, Antisense RNA and Co-expression of Sense and Antisense RNA on dEGFP Gene Expression

A human embryonic kidney cell line stably expressing the dEGFP gene under control of the cytomegalovirus immediate early promoter (and G418 resistant due to the presence of a linked Neo^{R} gene) was transfected with episomal plasmids that contained either the Hyg^{R} gene (conferring resistance to hygromycin) or the Pur^{R} gene (conferring resistance to puromycin) and sense and antisense expression cassettes. The structure of the cassettes used to express antisense complementary to the target mRNA or sense RNA homologous to the target mRNA are indicated in FIG. 1A. The ATG start codon in the sense gene was modified to prevent translation of the encoded sense RNA into dEGFP protein. Following co-transfection with the sense and antisense plasmids, cells containing both episomes and the target gene were selected using puromycin, hygromycin and G418. The control cells contained the two base vectors without antisense or sense genes, while the cells containing the antisense plasmid or sense plasmid only were co-transfected with the appropriate base vector containing the second selectable marker. In this way, all cells selected were resistant to puromycin, hygromycin and G418. After selection, all co-transfectants were subcultured, grown to different levels of confluence and analysed by FACs for their cell fluorescence profile. A summary of these results is indicated in FIG. 1B.

The results in FIG. 1B indicate that cells containing the vectors alone, the antisense plasmid alone or the sense plasmid alone did not display a reduction in dEGFP-mediated cell fluorescence. This outcome was observed using antisense or sense genes controlled by either the mammalian constitutive EF160 promoter or the Rous sarcoma virus LTR. In contrast, cells containing both the antisense and sense plasmids revealed an approximately 40% to 60% reduction in cell fluorescence controlled by the dEGFP target gene. The same trend was observed in cell cultures grown to three different degrees of confluence. These data show that co-expression of antisense and sense RNAs, in the presence of the target MnRNA, is more effective at suppressing the cellular phenotype associated with expression of the target gene in human cells than using an antisense or a sense plasmid alone. Thus, introducing two complementary RNAs, with the potential to form intermolecular dsRNA, into somatic human cells can regulate the expression of a specific gene in mammalian cell culture.

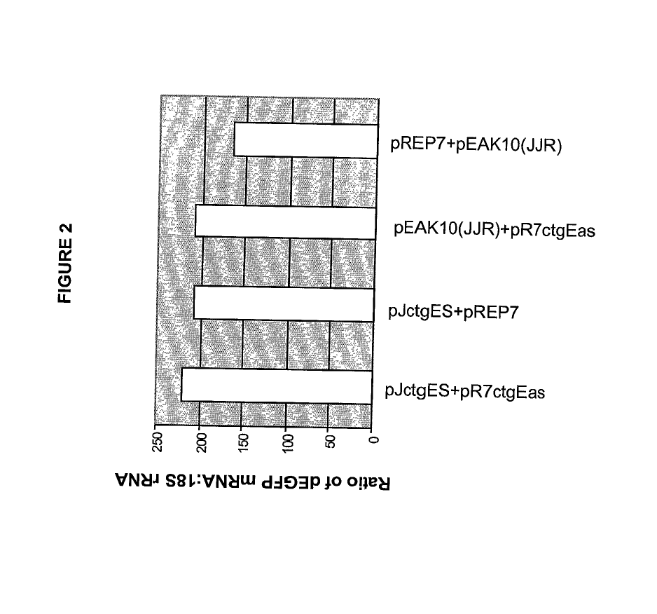

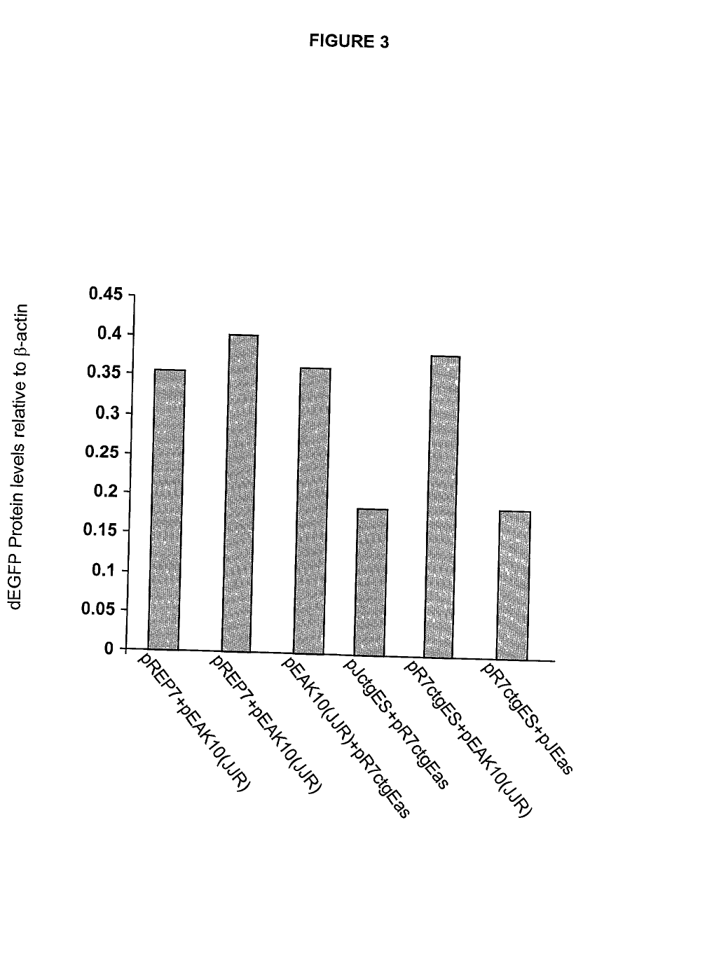

We further characterised the above transfectants for the effect of sense and antisense dEGFP RNA co-expression on the dEGFP target mRNA and protein steady-state levels. Northern analysis of total RNA isolated from control cells, antisense cells, sense cells and cells co-expressing sense and antisense RNAs showed that the all of these cell types displayed the same steady-state level of dEGFP target mRNA (FIG. 2). Therefore under these conditions, the reduction in dEGFP-mediated cell fluorescence in the antisense and sense RNA co-expressing cells did not appear to be due to increased turnover of the target mRNA. To examine the impact of co-expressing these complementary RNAs on the level of dEGFP protein, total protein was extracted from the above cells and analysed using dEGFP-specific and β-actin-specific antibodies (FIG. 3). Analysis of these protein blots indicated that the level of dEGFP protein was reduced by 50% in cells co-expressing antisense and sense RNA compared with all of the other cell types (FIG. 3B). This result shows that the observed phenotypic change in cell fluorescence in the antisense and sense co-expressing cells was due to the reduction in the steady-state level of the dEGFP protein. In addition, this suppressive effect was specific for the dEGFP protein and did not alter the steady-state level of either p53 or β-actin. Furthermore, the p53 protein was not activated since p21 protein levels were unchanged in the antisense and sense co-expressing cells in comparison to the cells containing the corresponding control vectors.

To demonstrate that the observed suppressive effect on dEGFP target gene expression required the co-expression of complementary RNAs, we examined the selected cells for the presence of the encoded RNAs. RT-PCR analysis indicated the presence of both the antisense and sense RNA-specific products. Furthermore, cloning and sequencing of these RT-PCR products showed that they were derived from the resident episomal plasmids and that the transcripts were not modified by adenosine deaminase activity. Overall, these results indicate that cells containing antisense and sense episomal plasmids expressed the two complementary dEGFP RNAs and that these RNAs were present in substochiometric steady-state levels relative to the target dEGFP mRNA.

The current models for gene interference via double-stranded RNA propose the generation of either small dsRNAs (called siRNAs) or small single-stranded RNAs (called stRNAs), both of which require the presence of the activity encoded by the enzyme Dicer. To determine whether the human homologue of Dicer was expressed in the human embryonic kidney cells selected to co-express the sense and antisense dEGFP RNAs, we performed RT-PCR on total RNA. Using methods described in example 1, a 579 base pair RT-PCR product was detected in cells co-expressing these two complementary RNAs. This indicated that these cells retained expression of one of the host-encoded proteins postulated to be involved in other forms of dsRNA-mediated gene regulation.

A common response of somatic mammalian cells to uptake of dsRNA is the activation of the PKR response that results in phosphorylation of PKR, general arrest of translation and eventually apoptosis. To examine whether co-expression of sense and antisense dEGFP RNAs, with the capacity to form dsRNA, affected the level of PKR we examined the steady-state level of this protein in control cells, antisense cells, sense cells and cells co-expressing sense and antisense RNAs. In all cell types tested, the level of the PKR protein remained unchanged and there was no evidence of a phosphorylated form of PKR. Thus, delivery of the sense and antisense dEGFP plasmids to the same cell resulted in a specific suppressive effect on dEGFP expression and, surprisingly, did not elicit a general cellular response to the presence of dsRNA, as might have been expected in light of the prior art.

### EXAMPLE 3

### Transferability of the dsRNA-mediated Suppression Effect to a Different Population of Cells Expressing Only the Target Gene

It has been noted in earlier studies using dsRNA as a mediator of gene inactivation in non-mammalian cells that a proportion of the suppressive effect can be transferred to other cells in vivo (Bosher and Labouesse, 2000) or in culture (Caplen et al., 2000). To examine the transferability of the dEGFP-specific dsRNA-mediated suppressive effect, we conducted a culture medium exchange experiment (FIG. 4A). Conditioned media from control cells and cells co-expressing antisense and sense dEGFP RNA was isolated and used to culture cells co-expressing antisense and sense dEGFP RNA and control cells, respectively. The addition of control medium to cells co-expressing antisense and sense dEGFP RNA did not alter the level of suppression of cell fluorescence (FIG. 4B). In contrast, control cells cultured in medium isolated from cells co-expressing antisense and sense dEGFP RNA displayed a reduction in dEGFP-mediated cell fluorescence. Western blot analyses of total protein from the recipient control cells indicated that only cells exposed to medium derived from the cells co-expressing sense and antisense dEGFP RNAs displayed a 50% reduction in dEGFP levels (FIG. 4C). No reduction in either p53 or β-actin steady-state levels was observed under these conditions in the recipient control cells. Thus, the suppressive effect generated within human cells by co-expressing sense and antisense RNA was transferable to cells that had not been previously exposed to either the sense or antisense RNAs.

### EXAMPLE 4

### The effect of Gene Constructs Expressing Intramolecular dsRNA Specific for dEGFP on Phenotypic Expression of the dEGFP Target Gene

Gene-specific dsRNA can be generated by either co-expressing two complementary RNA strands (discussed above) or using cassettes expressing RNAs with internal complementarity (referred to as inverted repeat plasmids), the latter of which express RNA capable of forming intramolecular dsRNA. A series of dEGFP-specific inverted repeat plasmids were constructed (FIG. 5A). Each of these plasmids was independently electroporated into dEGFP-expressing human cells and transfected cells identified by the RFP marker contained on the inverted repeat plasmids. This population was then assessed for the effect of inducing expression of the inverted repeat dsRNAs on dEGFP-mediated cell fluorescence (FIG. 5B). This analysis showed that at 48 hours post-electroporation (and 24 hours after the addition of ponasterone A to induce expression of the inverted repeat dsRNA), expression of the intramolecular dsRNA containing an internal intron reduced dEGFP-mediated cell fluorescence by ˜30% to 50%. This suppressive effect was more marked upon induction of expression of the conditional promoter controlling the inverted repeat cassettes. The appropriate length of the required inverted repeats can be determined by simple experimentation.

These results indicate that expression of inverted repeat dsRNAs can suppress phenotypic expression of a specific gene in human cultured cells.

### EXAMPLE 5

### Restricting the Expression of dsRNA to the Nucleus Using a cis-acting Ribozyme

One of the proposed limitations to using dsRNA to regulate gene expression in mammalian cells in the presence of a global response mechanism involving minimally PKR induction (Sharp, 1999). The result of activation of these activities is inhibition of cell growth and apoptosis. This general response is proposed to be restricted to the cell cytoplasm. It may be that some of the sense/antisense dsRNA in the experiments described herein is exported from the nucleus to the cytoplasm. As such, we have designed a strategy for avoiding the dsRNA-induced global response that involves expression of dsRNA in the nucleus. To this end, a DNA sequence encoding a cis-acting hammerhead ribozyme was introduced into pEAK(JJR)gfps between the GFP ORF and the poly A signal. The cis-acting ribozyme prevents polyadenylation and therefore blocks migration of the encoded transcript (dsRNA) to the cytoplasm (Liu et al., 1994). To test this concept, 293 cells were transfected with pEAK10(JJR)gfps, with or without the ribozyme, and fluorescence measured at 48 hrs and three weeks post-transfection.

The addition of the cis-acting ribozyme to the 3′ UTR of the GFP gene reduced fluorescence by 15%. Deletion of the polyadenylation (poly A) module from this construct resulted in cells showing 82% less fluorescence. One possible reason for this reduced expression of the GFP gene may have been the loss of the poly A tail and therefore transcript instability. However, a plasmid lacking both the cis ribozyme and the poly A sequences still expressed 98% cell fluorescence. The result obtained with the cis-acting ribozyme sequence, in the absence of the poly A signal, suggested the possibility that this construct would be more effective at retaining the encoded RNA (dsRNA) within the nucleus. It would be clear to one skilled in the art that this strategy, and the expression vectors described, could be used to retain two complementary RNAs and inverted repeat dsRNAs within the eukaryotic cell nucleus and thus further reduce the risk of induction of the PKR response.

Without being bound by theory, it is proposed that the mechanism of action of the present invention may be dependent on the actual formation of dsRNA. Further it is proposed that the dsRNA, once formed may be degraded to small fragments and that these fragments may interfere with translation from the mRNA of the target gene or nucleic acid sequence. However, it is of course possible that the mechanism of action is by other means which may or may not include one or more of these steps.

### EXAMPLE 6

### Testing of HIV-1-specific dsRNA Constructs in Mammalian Cells

One possible mechanism for dsRNA-mediated gene inhibition is highlighted in FIG. 7. This figure shows a proposed mechanism for dsRNA-mediated gene suppression, in which proteins bind to dsRNA and initiate cleavage, resulting in 21-23-mers. The protein-bound fragments then go through an amplification step (presumably by the implicated RNA polymerases) and hybridize to the target mRNA. Either the physical anti-sense block prevents transcription or, more likely, further proteins are sequestered and cleavage of the target RNA occurs.

The different ways of forming a dsRNA for specific gene suppression are illustrated in FIGS. 8A, B and C. The first mechanism (FIG. 8A) involves the cloning of an intervening sequence that, upon transcription, forms a loop as the complementary sequences bind. The second mechanism (FIG. 8B) involves the inclusion of an intron with a splice donor/splice acceptor site such that, upon transcription, the cell machinery will splice out the intron leaving a hairpin RNA molecule homologous to the target sequence. The third mechanism (FIG. 8C), which is a preferred embodiment of the present invention, involves the inclusion of an intervening sequence that is flanked by ribozymes such that, upon transcription, the ribozymes excise the intervening sequence, leaving a dsRNA that is homologous to the target mRNA.

To test whether HIV-1 replication is be blocked in human cells by dsRNA specific for regions of the HIV-1 viral RNA, the gene constructs outlined in FIG. 9 are constructed, all of which are in a Moloney Murine Leukemia Virus (MoMLV)-based, replication incompetent retroviral vector (LNL6). In the first construct (FIG. 9A) the following steps are performed: (a) a region of the HIV-1 genome encompassing nucleotides 530 to 1089 (of the HXB2 sequence) is cloned into LNL6 downstream of the Neo^{R} marker in the sense orientation relative to the 5′ LTR. This sequence is designated H5 and the sequence of the upper strand of this region is as follows:

| | |
|---|---|
| 5′AAGCTTGCCTTGAGTGCTTCAAGTAGTGTGTGCCCGTCTGTTGTGTGACTCT | (SEQ ID NO:31) |
| GGTAACTAGAGATCCCTCAGACCCTTTTAGTCAGTGTGGAAAATCTCTAGCA | |
| GTGGCGCCCGAACAGGGACCTGAAAGCGAAAGGGAAACCAGAGGAGCTCTC | |
| TCGACGCAGGACTCGGCTTGCTGAAGCGCGCACGGCAAGAGGCGAGGGGCG | |
| GCGACTGGTGAGTACGCCAAAAATTTTGACTAGCGGAGGCTAGAAGGAGAG | |
| AGATGGGTGCGAGAGCGTCAGTATTAAGCGGGGGAGAATTAGATCGATGGG | |
| AAAAAATTCGGTTAAGGCCAGGGGGAAAGAAAAAATATAAATTAAAACATA | |
| TAGTATGGGCAAGCAGGGAGCTAGAACGATTCGCAGTTAATCCTGGCCTGTT | |
| AGAAACATCAGAAGGCTGTAGACAAATACTGGGACAGCTACAACCATCCCT | |
| TCAGACAGGATCAGAAGAACTTAGATCATTATATATACAGTAGCAACCCTC | |
| TATTGTGTGCATCAAAGGATAGAGATAAAAGACACCAAGGAAGCT 3′ | |

(b) a region encoding an intervening sequence that is flanked by cis-acting ribozymes is then cloned downstream of H5. This fragment is composed of the following sequences and is designated IVSribozyme:

| | |
|---|---|
| 5′AGATCTGGCACTGAGTAATTGCTGCAGATCGTCAAAAGCAGAGTCCCTGA | (SEQ ID NO:32) |
| GTAGTCTCTAGCATACGGTACCTACTCAAGCTATGCATCAAGCTTGGTACCG | |
| AGCTCGGATCCACTAGTAACGGCCGCCAGTGTGCTGGAATTCGCCCTTAAGG | |
| GCGAATTCTGCAGATATCAAGCTTTCTAGAGTATGCTAGTAATGACGATCTG | |
| CAGCAATCTGATGAGTCCCTGAGGACGAAACTCAGTGCCAGATCT-3′ | |

(c) a region of the HIV-1 genome encompassing nucleotides 530 to 1089 (of the HXB2 sequence) is cloned downstream of the IVSribozyme in the antisense orientation relative to the 5′ LTR. This sequence is designated ASH5 and the sequence of the upper strand of this region is as follows:

| | |
|---|---|
| 5′AGCTTCCTTGGTGTCTTTTATCTCTATCCTTTGATGCACACAATAGAGGGTT | (SEQ ID NO:33) |
| GCTACTGTATTATATAATGATCTAAGTTCTTCTGATCCTGTCTGAAGGGATGG | |
| TTGTAGCTGTCCCAGTATTTGTCTACAGCCTTCTGATGTTTCTAACAGGCCAG | |
| GATTAACTGCGAATCGTTCTAGCTCCCTGCTTGCCCATACTATATGTTTTAAT | |
| TTATATTTTTTCTTTCCCCCTGGCCTTAACCGAATTTTTTCCCATCGATCTAAT | |
| TCTCCCCCGCTTAATACTGACGCTCTCGCACCCATCTCTCTCCTTCTAGCCTC | |
| CGCTAGTCAAAATTTTTGGCGTACTCACCAGTCGCCGCCCCTCGCCTCTTGCC | |
| GTGCGCGCTTCAGCAAGCCGAGTCCTGCGTCGAGAGAGCTCCTCTGGTTTCC | |
| CTTTCGCTTTCAGGTCCCTGTTCGGGCGCCACTGCTAGAGATTTTCCACACTG | |
| ACTAAAAGGGTCTGAGGGATCTCTAGTTACCAGAGTCACACAACAGACGGG | |
| CACACACTACTTGAAGCACTCAAGGCAAGCTT 3′ | |

For the second construct (FIG. 9B), a 61 base pair TAR sequence of HIV-1 (the HIV R region) is cloned into the U5 region of the 5′ LTR of the first construct described above, which would permit Tat to enhance transcription. The sequence of the upper strand of the HIV R region sequence is as follows (GenBank accession number K03455):

| | |
|---|---|
| 5′GGGTCTCTCTGGTTAGACCAGATCTGAGCCTGGGAGCTCTCTGGCTAACTA | (SEQ ID NO:34) |
| GGGAACCCACTGCTTAAGCCTCAATAAAGCTTGCCTTGAGTGCTTCA3′ | |

For the third construct (FIG. 9C), the nucleotide sequence encoding the Tat protein is cloned in place of the NeoR marker in the first construct, and the SV40-driven NeoR marker from pLXSN (Clontech, USA) is subdloned downstream of the ASH5 sequence. The amino acid sequence of the Tat protein included in this construct is as follows:

N-met-glu-pro-val-asp-pro-arg-leu-glu-pro-trp-lys-his-pro-gly-ser-gln-pro-lys-tbr-ala-cys-thr-asn-cys-tyr-cys-lys-lys-cys-cys-phe-his-cys-gln-val-cys-phe-ile-thr-lys-ala-leu-gly-ile-ser-tyr-gly-arg-lys-lys-arg-gln-arg-arg-arg-pro-pro-gln-gly-ser-gln-thr-his-gln-val-ser-leu-ser-lys-gln-pro-thr-ser-gln-ser-arg-gly-asp-pro-thr-gly-pro-lys-glu-C (SEQ ID NO:35)

Each of these three constructs is introduced into CEM-T4 cells via infection with the retrovirus containing the constructs designated in FIG. 9. Following selection in G418, these cells are then challenged with HTLV-IIIB, and then at days 5, 6 and/or 7 post-infection cell superuatants are assayed for p24 antigen (using the Innotest, Innunogenetics, Belgium) to assess the impact on HIV replication.

### EXAMPLE 7

### Treatment of HIV Patients Using the dsRNA-encoding Retroviral Constructs

The treatment includes the mobilisation of hematopoietic progenitor cells (CD34+ cells) by Granulocyte-Colony Stimulating Factor (G-CSF), from the bone marrow and collection by apheresis. CD34+ cells are enriched and cultured ex vivo by methods well known in the art. The CD34+ cells are transduced with replication incompetent retrovirus containing constructs described in Example 6 and encoding dsRNA before being reinfused back into the patient. The dsRNA containing CD34+ cells then migrate to the bone marrow and in time contribute to the peripheral lymphocyte population. The dsRNA offers protection from HIV infection and a reduced amount of viral production within infected cells.

Although the invention has been described with reference to specific examples, it will be clear to those skilled in the art that the invention may be embodied in many other forms.

### REFERENCES

Bosher, J. M., Laboueusse, M., 2000. RNA interference: genetic wand and genetic watchdog. Nature Cell Biol. 2, E31-E36.

Bunnell, B. A., Fillmore, H., Gregory, P., Kidd, V. J., 1990. A dominant negative mutation created by ectopic expression of an AU-rich 3′ untranslated region. Somatic Cell Mol. Genet. 16,151-162.

Caplen, N. J., Fleenor, J., Fire, A., Morgan, R. A., 2000. DsRNA-mediated gene silencing in cultured Drosophila cells: a tissue culture model for the analysis of RNA interference. Gene 252, 95-105.

Clemens, M. J., 1997. PKR—a protein kinase regulated by double-stranded RNA. Int. J. Biochem. Cell. Biol. 29, 945-949.

Chehab, F. F. et al. 1987 Nature 329:293-294.

Cuenoud, B. and Szostak, J. W. 1995 Nature 375:611-614.

Eckner, R. Ellmeier, W. and Bimstiel, M. L. (1991) Mature mRNA 3′ end formation stimulates RNA export from the nucleus. EMBO 10(1 1):3513-3522.

Fahy, E., et al. (1991) PCR Methods Appl 1: 25-33.

Fields, S., 1997. The future is function. Nature Genet. 15, 325-327.

Fire, A., Xu, S., Montgomery, M. K., Kostas, S. A., Driver, S. E., Mello, C. C., 1998. Potent and specific interference by double-stranded RNA in Caenorhabditis elegans. Nature 391, 806-811.

Herskowitz, I., 1987. Functional inactivation of genes by dominant negative mutations. Nature 329, 219-222.

Jonas, V., et al. (1993) Journal of Clinical Microbiology 31:2410-2416.

Kramvis, A., et al. (1996) Journal of Clinical Microbiology 34: 2731-2733.

Liu, Z., Batt, D. B., Carmichael, G. G., 1994. Targeted nuclear antisense RNA mimics natural antisense-induced degradation of polyoma virus early RNA. Proc. Natl. Acad. Sci. USA 91, 4258-4262.

Marcus, P. I., 1983. Interferon induction by viruses: one molecule of double-stranded RNA as the threshold for interferon induction. Interferon 5, 115-180.

No, D., Yao, T., Evans, R. M., 1996. Ecdysone-inducible gene expression in mammalian cells and transgenic mice. Proc. Natl. Acad. Sci. USA 93, 3346-3351.

Saez, E., Nelson, M. C., Eshelman, B., Banayo, E., Koder, A., Cho, G. J., Evans, R. M., 2000. Identification of ligands and coligands for the ecdysone-regulated gene switch. Proc. Natl. Acad. Sci. USA 97, 14512-14517.

Saiki, R. K. et al. (1985) Science 230:1350-1354.

Sanford, J. C., 1988. Applying the PDR principle to AIDS. J. Theor. Biol. 130, 469-480.

Sambrook, J., Fritsch, E. F., Maniatis, T., 1989. Molecular Cloning: A Laboratory Manual. Cold Spring Harbor Press, Cold Spring Harbor, N.Y.

Sharp, P. A., 1999. RNAi and double-stranded RNA. Genes Develop. 13, 139-141.

Sun, L.-Q., et al (2000) Pharmacological Reviews 52:325-347.

Van der Krol, A. R., Mol, J. N., Stuitje, A. R., 1988. Modulation of eukaryotic gene expression by complementary RNA and DNA sequences. Biotechniques 6, 958-976.

Walder, R. Y., et al. (1993) Nuclei Acid Research 21(18):4339-4343.

Walker, G. T., et al. (1992) Nuclei Acids Res. 20:1691.

Yong, S. L., et al. (1995) Nuclei Acids Research 23:1640.

What is claimed is:

1. A double-stranded RNA complex comprising:
(c) a first ribonucleic acid molecule capable of hybridizing under physiological conditions to at least a portion of an mRNA molecule; and
(d) a second ribonucleic acid molecule wherein at least a portion of the second ribonucleic acid molecule is capable of hybridizing under physiological conditions to the first portion.

2. A RNA complex of claim 1 wherein the first and second portions are separate ribonucleic acid molecules.

3. A double-stranded RNA molecule of claim 1 wherein the mRNA is encoded by a gene in a cell.

4. A linear RNA molecule capable of forming a dsRNA complex wherein the RNA molecule comprises:
(c) a first portion that hybridizes to at least a portion of a mRNA molecule; and
(d) a second portion wherein at least part of the second portion is capable of hybridizing to the first portion to form a hairpin dsRNA complex.

5. A double-stranded RNA molecule of claim 4 wherein the mRNA is encoded by a gene in a cell.

6. A linear RNA molecule of claim 4 further comprising a third portion of ribonucleic acid interposed between the first and second portions.

7. A linear RNA molecule of claim 6 wherein the third portion promotes hybridization between the first and second portion.

8. A RNA molecule of claims 1 or 4 further comprising an additional RNA portion of ribonucleic acid that enhances the ability of dsRNA to alter transcription from the gene encoding the mRNA molecule.

9. A RNA molecule of claim 8 wherein the additional RNA portion encodes an RNA molecule.

10. A RNA molecule of claim 8 wherein the additional RNA portion encodes a protein.

11. A RNA of claim 10 wherein the protein is Tat.

12. A RNA of claim 6 wherein the third portion of ribonucleic acid further comprises at least one ribozyme and a target sequence recognizable by the ribozyme wherein the target sequence is not present in the first portion and the second portion.

13. A RNA of claim 12 wherein the double-stranded RNA complex is formed upon hybridization of the first and second portion and the target sequence is cleaved by the hairpin dsRNA.

14. A RNA of claim 6 wherein the third portion of ribonucleic acid further comprises an intron or a linker sequence.

15. A linear RNA molecule capable of forming a dsRNA complex wherein the RNA molecule comprises:
(d) a first portion that comprises a region of RNA that is complementary to at least a portion of a mRNA molecule encoded by a gene;
(e) a second portion capable of hybridizing to at least part of the first portion; and
(f) a third portion positioned between the first and second portions to permit the first and second portions to hybridize with one another.

16. A linear RNA molecule of claim 15 wherein the third portion comprises at least one ribozyme and a target sequence recognized by the ribozyme wherein the target sequence is not present in the first or second portion.

17. A linear RNA molecule of claim 15 wherein second sequence comprises a polyadenylation signal.

18. A linear RNA molecule of claim 15 wherein the third portion comprises a plurality of ribozymes and target sequences capable of cleavage thereby.

19. A linear RNA molecule capable of forming a dsRNA complex wherein the RNA molecule comprises:
(c) a first portion that hybridizes to at least a portion of a mRNA molecule encoded by a gene; and
(d) a second portion wherein at least part of the second portion is capable of hybridizing to the first portion and wherein the second portion comprises a polyadenylation signal and a ribozyme positioned between the part of the second portion capable of hybridizing to the first portion and the polyadenylation signal
wherein the ribozyme is capable of removing the polyadenylation signal.

20. A linear RNA molecule of claim 19 wherein the ribozyme is a cis-acting hammerhead ribozyme.

21. At least one DNA molecule encoding the RNA molecules of any of claims 1-20.

22. A DNA molecule of claim 21 wherein a single DNA molecule encodes the RNA molecules of any one of claims 1-20.

23. A DNA molecule of claim 21 wherein two DNA molecules encode the RNA molecules of claim 1.

24. A eukaryotic cell comprising the RNA molecules of claims 1-20.

25. A eukaryotic cell comprising the DNA of molecules of any of claims 21-23.

26. A eukaryotic cell of claims 24 or 25 wherein the cell is a mammalian cell.

27. A eukaryotic cell of any of claims 24-26 wherein the cell is a human cell.

28. A cell of claim 27 wherein the cell further comprises HIV nucleic acid.

29. A cell of any of claims 24-27 wherein the cell is a neoplastic cell.

30. A vector encoding at least one of the RNA molecules of any of claims 1-20.

31. A vector comprising the DNA of any of claims 21-23.

32. A vector of claim 30 or 31 wherein the vector is a plasmid, an adenovirus, an adenoassociated virus, or a retrovirus.

33. A vector of claim 32 wherein the plasmid is an episomal plasmid.

34. A method for inhibiting protein expression in a eukaryotic cell comprising the step of introducing the RNA of any of claims 1-20, the DNA molecules of claims 21-23 or the vectors of claims 30-32 into the cell.

35. A method of claim 34 wherein the eukaryotic cell is a mammalian cell.

36. A method of claim 35 wherein the cell is a human cell, a somatic cell, an undifferentiated, dedifferentiated, neoplastic cell or a chimeric cell.

37. A method of claim 34 wherein the RNA, DNA is introduced into the cell using a vesicle or is delivered by microinjection.

38. A method of claim 34 wherein the mRNA is selected from the group consisting of a cancer-related gene, a rheumatoid arthritis-related gene and a viral gene.

39. A method of claim 38 wherein the mRNA is an HIV-derived gene.

40. A method of claim 39 wherein the gene is selected from the group consisting of tat, nef, rev, ma, ca, ne, pg vpu, pr, vif su, tm, vpr, rt and in.

41. A method of inhibiting protein expression from a gene in a cell comprising the step of;
introducing a linear RNA molecule capable of forming a dsRNA complex into a cell, wherein the RNA molecule comprises:
(a) a first portion that hybridizes to at least part of a mRNA molecule encoded by a gene; and
(b) a second portion wherein at least part of the second portion is capable of hybridizing to the first portion

42. A method of claim 41 wherein the second portion comprises a polyadenylation signal positioned at the 3′ end of the linear RNA molecule.

43. A method of claim 42 wherein the second portion further comprises a ribozyme positioned between the part of the second portion capable of hybridizing to the first portion and the polyadenylation signal, wherein the ribozyme is capable of removing the polyadenylation signal.

44. A method of claim 43 wherein the ribozyme is a cis-acting hammerhead ribozyme.

45. A method of claim 41 wherein the cell is a mammalian cell.

46. A method of claim 41 wherein the cell is in vitro.

47. A method of claim 41 wherein the cell is in vivo.

48. A method of claim 41 wherein the introducing step employs microinjection.

49. A method of claim 41 wherein the RNA is encoded by a DNA molecule and the DNA molecule is transcribed in the cell.

50. A method of claim 41 wherein the RNA is introduced as a vector.

51. A method of claim 50 wherein the vector is RNA or DNA.

52. A method of claim 51 wherein the vector is a plasmid, adenovirus, adeno-associated virus or a retrovirus.

53. A method of claim 41 wherein the RNA is synthesized outside the cell.

54. A method of claim 41 wherein the RNA is synthesized inside the cell.

55. A method of claim 43 wherein the RNA is retained in the nucleus.

56. A method for localizing a dsRNA molecule to the nucleus of a cell comprising the step of:
introducing a DNA molecule encoding a linear RNA molecule capable of forming a dsRNA complex into a cell wherein the RNA molecule encoded by the DNA molecule comprises:
(a) a first portion that hybridizes to at least a portion of a mRNA molecule encoded by a gene; and
(b) a second portion wherein at least part of the second portion is capable of hybridizing to the first portion and wherein the second portion comprises a polyadenylation signal and a ribozyme positioned between the part of the second portion capable of hybridizing to the first portion and the polyadenylation signal,
wherein the ribozyme is capable of removing the polyadenylation signal thereby retaining the RNA in the nucleus.

57. The method of claim 56 wherein the ribozyme is a cis-acting hammerhead ribozyme.

58. A method for modulating expression of a nucleic acid sequence in a cell comprising exposing the cell to culture medium in which cells comprising a dsRNA complex comprising a first portion that hybridizes to at least part of a mRNA molecule encoded by a gene; and a second portion wherein at least part of the second portion is capable of hybridizing to the first portion have been maintained in cell culture.

59. A method of identifying the function of a gene in a cell comprising the step of;
(a) binding a dsRNA molecule to an mRNA molecule in a cell wherein the dsRNA molecule comprises a first ribonucleic acid molecule capable of hybridizing under physiological conditions to at least a portion of an mRNA molecule; and a second ribonucleic acid molecule wherein at least a portion of the second ribonucleic acid molecule is capable of hybridizing under physiological conditions to the first portion; and
(b) detecting a change in the cell resulting from the binding.

60. A method of forming a double-stranded RNA in a cell comprising the step of introducing the RNA molecule of any of claims 1-20 or the DNA molecule of claims 21-23 into a cell.

61. A composition for inhibiting the expression of a gene in a eukaryotic cell comprising:
a RNA molecule of claims 1 or 4 wherein the RNA molecule further comprises an additional RNA portion of ribonucleic acid that enhances the ability of dsRNA to alter transcription from the gene encoding the mRNA molecule.

61. The composition of claim 61 further comprising a third portion of ribonucleic acid interposed between the first and second portions wherein the third portion promotes hybridization between the first and second portion.

62. Use of any of the RNA of claims 1-20, the DNA of claims 21-23 or the vectors of claims 31-33 to inhibit expression of a gene in a cell.

63. A pharmaceutical composition comprising the RNA of claims 1-20, the DNA of claims 21-23 or the vectors of claims 31-33.

64. A microinjection apparatus comprising a pharmaceutical composition comprising the RNA of claims 1-20, the DNA of claims 21-23 or the vectors of claims 31-33.

65. A lipid vesicle comprising the RNA of claims 1-20, the DNA of claims 21-23 or the vectors of claims 31-33.

66. Use of any of the RNA of claims 1-20, the DNA of claims 21-23 or the vectors of claims 31-33 to determine the functions of genomic nucleic acid or viral nucleic acid in a cell.


SEQUENCE LISTING (35 sequences)

SEQ ID NO: 1 - DNA, 34 bp, Artificial Sequence
  PCR primer
tgaggattcaccggtcgccaccctggtgagcaag

SEQ ID NO: 2 - DNA, 33 bp, Artificial Sequence
  PCR Primer
tgaggattcacaaaccacaactagaatgcagtg

SEQ ID NO: 3 - DNA, 24 bp, Artificial Sequence
  PCR Primer
tcagatccgctagcgctaccggac

SEQ ID NO: 4 - DNA, 24 bp, Artificial Sequence
  PCR primer
acaaaccacaactagaatgcagtg

SEQ ID NO: 5 - DNA, 29 bp, Artificial Sequence
  PCR Primer
tctctagggatcctcagtcagtcaggatg

SEQ ID NO: 6 - DNA, 32 bp, Artificial Sequence
  PCR primer
catcctgactgactgaggatccctagagaata

SEQ ID NO: 7 - DNA, 34 bp, Artificial Sequence
  PCR primer
tgaagatctaccggtcgccaccctggtgagcaag

SEQ ID NO: 8 - DNA, 33 bp, Artificial Sequence
  PCR primer
tgagaattcacaaaccacaactagaatgcagtg

SEQ ID NO: 9 - DNA, 30 bp, Artificial Sequence
  PCR primer
tgaactagttctcggccgcatattaagtgc

SEQ ID NO: 10 - DNA, 24 bp, Artificial Sequence
  PCR primer
tgaaagcttaagtttaaacgctag

SEQ ID NO: 11 - DNA, 31 bp, Artificial Sequence
  PCR primer
gcgcactagtcgtattaccgccatgcattag

SEQ ID NO: 12 - DNA, 31 bp, Artificial Sequence
  PCR primer
gcgcactagtacgccttaagatacattgatg

SEQ ID NO: 13 - DNA, 31 bp, Artificial Sequence
  PCR primer
gcgcagatctaccggtcgccaccctggtgag

SEQ ID NO: 14 - DNA, 30 bp, Artificial Sequence
  PCR primer
gcgcgaattccatctacacattgatcctag

SEQ ID NO: 15 - DNA, 41 bp, Artificial Sequence
  PCR primer
tgagaattcagatctaccggtcgccaccctggttgagcaag

SEQ ID NO: 16 - DNA, 33 bp, Artificial Sequence
  PCR primer
tgagaattccttcacctcggcgcgggtcttgta

SEQ ID NO: 17 - DNA, 31 bp, Artificial Sequence; identical to SEQ ID NO: 13
  PCR primer

SEQ ID NO: 18 - DNA, 30 bp, Artificial Sequence
  PCR primer
gcgcagatctcatctacacattgatcctag

SEQ ID NO: 19 - DNA, 31 bp, Artificial Sequence
  PCR primer
gcgcggtaccgaattaattcgctgtctgcga

SEQ ID NO: 20 - DNA, 30 bp, Artificial Sequence
  PCR primer
gcgcggtacccgacctgcacttggacctgg

SEQ ID NO: 21 - DNA, 33 bp, Artificial Sequence
  PCR primer
tgaaagcttgccgccaccatgagcaagggcgag

SEQ ID NO: 22 - DNA, 33 bp, Artificial Sequence
  PCR primer
tgaaagctttcacttgtacagctcgtccatgcc

SEQ ID NO: 23 - DNA, 97 bp, Artificial Sequence
  Oligonucleotide
gaattcaattcggcccttatcagggccatgcatgtcgcggccgcctccgcggccgcctgatgagtccgtgaggacgaaacatgcatagggccctgat

SEQ ID NO: 24 - DNA, 91 bp, Artificial Sequence
  Oligonucleotide
atcgggccctatgcatgtttcgtcctcacggactcatcaggcggccgcggaggcggccgcgacatgcatggccctgataagggccgaattg

SEQ ID NO: 25 - DNA, 21 bp, Artificial Sequence
  PCR primer
gcaattgaaccggtgcctaga

SEQ ID NO: 26 - DNA, 18 bp, Artificial Sequence
  PCR primer
gaacttgtggccgtttac

SEQ ID NO: 27 - DNA, 21 bp, Artificial Sequence
  PCR primer
cgcagatcctgagcttgtatg

SEQ ID NO: 28 - DNA, 18 bp, Artificial Sequence
  PCR primer
cactgcattctagttgtg

SEQ ID NO: 29 - DNA, 25 bp, Artificial Sequence
  PCR primer
ttaaccagctgtggggagagggctg

SEQ ID NO: 30 - DNA, 25 bp, Artificial Sequence
  primer
agccagcgatgcaaagatggtgttg

SEQ ID NO: 31 - DNA, 559 bp, Artificial Sequence
  region of the HIV-1 genome
aagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagct

SEQ ID NO: 32 - DNA, 252 bp, Artificial Sequence
  IVS ribozyme
agatctggcactgagtaattgctgcagatcgtcaaaagcaggagtccctgagtagtctctagcatacggtacctactcaagctatgcatcaagcttggtaccgagctcggatccactagtaacggccgccagtgtgctggaattcgcccttaagggcgaattctgcagatatcaagctttctagagtatgctagtaatgacgatctgcagcaatctgatgagtccctgaggacgaaactcagtgccagatct

SEQ ID NO: 33 - DNA, 559 bp, Artificial Sequence
  fragment with antisense orientation of IVS ribozyme
agcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaggtccctgttcgggcgccactgctagagattttccacactgactaaaagggtctgagggatctctagttaccagagtcacacaacagacgggcacacactacttgaagcactcaaggcaagctt

SEQ ID NO: 34 - DNA, 98 bp, Artificial Sequence
  region containing HIV Tar sequence
gggtctctctggttagaccagatctgagcctgggagctctctggctaactagggaacccactgcttaagcctcaataaagcttgccttgagtgcttca

SEQ ID NO: 35 - protein, 86 aa, Artificial Sequence
  HIV-1 Tat amino acid sequence
MEPVDPRLEPWKHPGSQPKTACTNCYCKKCCFHCQVCFITKALGISYGRKKRRQRRRPPQGSQTHQVSLSKQPTSQSRGDPTGPKE